(19) United States
(12) Patent Application Publication
(10) Pub. No.: US 2019/0315878 A1
(43) Pub. Date: Oct. 17, 2019

(54) BISPECIFIC ANTIBODIES THAT MODULATE TLR-4 SIGNALING AND USES THEREOF

(71) Applicant: ARGOS THERAPEUTICS INC., Durham, NC (US)

(72) Inventors: Charles NICOLETTE, Durham, NC (US); Mark DEBENEDETTE, Durham, NC (US); Joseph HORVATINOVICH, Raleigh, NC (US)

(21) Appl. No.: 16/347,678

(22) PCT Filed: Nov. 3, 2017
(86) PCT No.: PCT/US17/59851

§ 371 Date: May 6, 2019

Related U.S. Application Data
(60) Provisional application No. 62/418,713, filed on Nov. 7, 2016.

(51) Int. Cl. C07K 16/28; C07K 16/18; A61P 37/06

(52) U.S. Cl. CPC C07K 16/2884; C07K 16/18; C07K 2317/76; C07K 2317/31; A61P 37/06

(57) ABSTRACT

Antibodies are provided that alter the TLR4 signaling pathway to produce an immunosuppressive effect. The antibodies are useful for the treatment or prevention of an unwanted immune response in a subject, such as autoimmune disease, transplant rejection, and allergic reactions.

### FIELD OF THE INVENTION

The invention relates to antibodies that are capable of modulating the response of the IRAK pathway to signals from TLR-4 complexes and the use of such antibodies in the treatment or prevention of immune-related diseases and disorders such as allergy, autoimmune disease and transplant rejection.

### BACKGROUND OF THE INVENTION

Pathogen-Associated Molecular Patterns (“PAMPs”) are molecules associated with groups of pathogens that serve as strong inducers of innate immunity. PAMPs include bacterial lipopolysaccharides (“LPSs”), endotoxins found on the cell membranes of bacteria which are considered to be the prototypical class of PAMPs. LPSs are specifically recognised by Toll-like Receptor 4 (TLR4), a recognition receptor of the innate immune system, and provoke an inflammatory response (see Park et al. (2009) Nature 458: 1191-1195; Takeda and Akira (2001) Jpn. J. Infect. Dis 54: 209-19; Kirschning and Bauer (2001) Int. J. Med. Microbiol. 291: 251-60; Beutler (2002) Curr. Top. Microbiol. Immunol 270: 109-20).

TLR4 does not bind LPS directly; rather, LPS signaling through TLR4 requires the co-receptors CD14 and MD-2 (Park and Lee (2013) Exp. Mol. Med 45: e66; Triantafilou et al. 25 (2004) Biochem. J. 381: 527-36; da Silva et al. (2001) J Biol. Chem 276: 21129-35). LPS first binds CD14, which transfers LPS to MD-2. Once bound to LPS, MD-2 is responsible for the dimerization of TLR4 molecules (Park et al. (2009) Nature 458: 1191-1195; Kim et al. (2007) Cell 130: 906-917). The crystal structure of the TLR4/MD-2/LPS complex reveals that LPS binds to a hydrophobic pocket within MD-2 and alters the heterodimerized TLR4 complex (Park et al. (2009) Nature 458: 1191-95, Park and Lee (2013) Exp. Mol. Med 45: e66, Triantafilou et al. (2004) Biochem. J. 381: 527-36). The ligand-induced dimerization of TLR4 results in the recruitment of MyD88 and autophosphorylation of the IL-1 receptor associated kinase family members IRAK-1 and IRAK-4, which triggers NF-kB activation via TRAF6 (Bode et al. (2012) Cell Signal 24: 1185-94, Ringwood and Li (2008) Cytokine 42: 1-7, O'Neill (2008) Immunity 29: 12-20).

Altering TLR4 signaling can attenuate the LPS/TLR4 pro-inflammatory cascade, but the underlying molecular mechanism was previously unknown. Here, Applicants disclose for the first time the molecular mechanism of action of sCD83 and identify the key molecular entities that are essential for sCD83 activity, thus providing a framework for both activating and interfering with this pathway using bispecific antibodies. The present invention addresses the previous problems in the art of how to utilize the sCD83 pathway for therapeutic benefit.

### BRIEF DESCRIPTION OF THE FIGURES

FIGS. 1A, 1B, 1C, and 1D: Flow cytometry analysis of cell surface binding of sCD83 protein. sCD83 protein was added to PBMCs, and binding to CD14-positive monocytes was detected using a secondary goat anti-human CD83 biotinylated antibody followed by APC-Streptavidin. In FIG. 1A, background cell staining is shown with anti-human CD83 biotinylated antibody followed by APC-Streptavidin in the absence of sCD83 protein. FIG. 1B shows results from sCD83 binding to the cell surface of monocytes and detected with goat anti-human CD83 biotinylated antibody followed by APC-Streptavidin. For the data shown in FIG. 1C, cells were simultaneously double stained with both the Alexa Fluor® 488-dye-conjugated sCD83 protein and the unlabeled protein detected with the anti-CD83 antibody followed by APC-Streptavidin. For the data shown in FIG. 1D, to generate a competition binding curve, cells were pre-incubated with varying concentrations (0.25 μg to 4 μg) of unconjugated sCD83 protein for 15 minutes at room temperature. After incubation, Alexa Fluor® 488-dye-labeled sCD83 was added to the cell suspension without prior washing. The percentage of Alexa Fluor® 488-dye-labeled sCD83 binding is shown in the presence of increased concentrations of unlabeled sCD83. Data shown are representative of 2-5 experiments.

FIGS. 2A, 2B, 2C, 2D, 2E, and 2F: sCD83 binds to CD14+ dendritic cells present in the peripheral blood. FIG. 2A shows flow cytometry analysis of plasmacytoid DCs in peripheral blood identified by expression of CD123 and CD303. For data shown in FIG. 2B, CD14+ DCs within the CD123+/CD303+ plasmacytoid DC subset were stained for CD14 expression and sCD83 protein binding was detected as described in the FIG. 1 legend. FIG. 2C shows background staining in the absence of sCD83 protein. FIG. 2D shows flow cytometry analysis of myeloid DCs in the peripheral blood identified by expression of CD1c and CD2. For the data shown in FIG. 2E, CD14+ DCs within the CD1c+/CD2+ myeloid DC subset were stained for CD14 expression and sCD83 protein binding was detected as described above in the FIG. 1 legend. FIG. 2F shows background staining in the absence of sCD83 protein.

FIGS. 3A, 3B, and 3C: sCD83 binds to monocytes expressing CD14/TLR4/MD-2 CD44 co-receptors. FIG. 3A shows sCD83 protein binding to Donor 1 PBMCs while FIG. 3B shows CD83 protein binding to Donor 2 PBMCs. Binding was determined post thaw as described above (first panel). In addition, expression of CD14 (second panel), TLR4 (third panel) and MD-2 (fourth panel) was determined. FIG. 3C shows results of experiments in which PBMCs were incubated overnight in GM-CSF and MD-2 protein expression (i) and sCD83 binding (ii) were measured. CD44v6 expression was determined on post thaw cells (iii), or after overnight culturing in GM-CSF (iv). Data shown are representative of 3 experiments.

FIGS. 4A, 4B, 4C, and 4D: Blocking sCD83 binding in the presence of antibodies to CD14, MD-2, TLR4 and CD44. FIG. 4A: sCD83 binding was determined on CD14+ monocytes as described in FIG. 1 in the presence of two isotype control IgG antibodies. FIG. 4B: Anti-CD14 antibody clones M5E2, My4, or 61D3 were incubated with PBMCs prior to the addition of sCD83 protein, and sCD83 binding was determined. FIG. 4C: Anti-MD-2 antibody clones 9B4 and 288307 and anti-TLR4/MD-2 antibody clone MTS510 were incubated with PBMCs prior to the addition of sCD83 protein and sCD83 binding was determined. FIG. 4D: Anti-CD44s antibody clones 515, 153C1, and 5F12 were incubated with PBMCs prior to the addition of sCD83 protein and sCD83 binding was determined. Data shown are representative of 3 experiments.

FIGS. 5A, 5B, and 5C: MD-2 enhances sCD83 binding to monocytes. FIG. 5A: The percent maximum sCD83 protein binding was determined in the presence of anti-CD44s antibody (clone IM-7) or with anti-CD44s antibody+soluble MD-2 protein. Cells were incubated sequentially with anti-CD44 antibody then with sCD83 and MD-2 proteins pre-mixed prior to addition to cells. FIG. 5B: sCD83 binding (thick histogram) on the cell surface in the presence of anti-CD44v6 antibody (clone VFF-7) (open histogram). Shade histogram shows staining in the absence of sCD83 protein. FIG. 5C: PBMCs were electroporated without RNA (i), or with CD14 RNA (ii), MD-2 RNA (iii) or TLR4 RNA (iv), and sCD83 binding to gated monocytes was determined as described. Shaded histograms represent staining with biotinylated anti-CD83 antibody and streptavidin-APC in the absence of sCD83. Open histograms represent staining in the presence of sCD83 protein. Percent positive sCD83 binding and the Mean Fluorescence Intensity (MFI) of sCD83 binding is shown.

FIGS. 6A and 6B: sCD83 binds to MD-2 with high affinity near the KDO-lipid A binding site on TLR4/MD-2. FIG. 6A: Recombinant MD-2 (♦), CD14 (▪) or CD44 proteins (▴) at the indicated concentrations were added to sCD83 coated plates and bound protein was detected using biotin conjugated antibodies to MD-2, CD14 or CD44 respectively. Non-specific binding determined using plates not coated with sCD83 was subtracted from specific binding detection. FIG. 6B: MD-2 protein at 0.25 μg/mL was co-incubated with serial dilutions of LPS (♦), KDO-lipid A (▪), Lipid A diphosphate (●), or Lipid A monophosphate (▴) prior to addition to sCD83 coated plates. Alternatively, serial dilutions of KDO-lipid A (□) were added first to the sCD83 coated plates and plates were washed prior to the addition of MD-2 protein. Biotinylated anti-MD-2 antibody was used to detect MD-2 protein bound to immobilized sCD83. Binding affinity for MD-2 was calculated using a five-parameter logistic curve fitting within SigmaPlot® statistical analysis (Systat Software, Inc., San Jose, Calif.). Data are representative of two experiments.

FIGS. 7A, 7B, 7C, 7D, and 7E: sCD83 signals through TLR4/MD-2 co-receptor by down regulating IRAK-1 expression. Purified monocytes were cultured overnight in medium containing GM-CSF and then stimulated with 25 μg/ml sCD83 protein or 100 ng/ml LPS for: 60 minutes (shown in FIG. 7A); 10, 15 or 30 minutes (shown in FIG. 7B); 24, 48 or 72 hours (shown in FIG. 7C); 6, 24, 72, and 7 days (shown in FIG. 7D); or 30 minutes or 2 or 6 hours (shown in FIG. 7E). Cell lysates were prepared and equivalent cellular protein concentrations were Western blotted with antibodies to IRAK-1, IRAK-2, IRAK-M, IRAK-4, TRAF6 or β-actin as a control for loading. Data are representative of 3 experiments.

FIGS. 8A, 8B, 8C, 8D, and 8E: sCD83 blocks T cell proliferation and renders T cells unresponsive to IL-2. CFSE-labeled PBMCs were stimulated with anti-CD3 and anti-CD28 antibodies for six days with or without the addition of sCD83 protein. Proliferation was measured by CFSE dilution using flow cytometry. FIG. 8A: Dot plots showing gating of CFSE low CD4+ T cells and CD8+ T cells representing cells that proliferated in the presence or absence of sCD83 protein. FIG. 8B: Cells were treated with 0, 2, 10 or 50 μg/mL sCD83 and the total number of proliferating CD4+ T cells (closed bars) and CD8+ T cells (open bars) were calculated. FIG. 8C: Eight days post T cell stimulation, 20 U/mL IL-2 was added to PBMC cultures previously stimulated with anti-CD3/anti-CD28 antibodies either in the presence (open symbols) or absence of sCD83 protein (closed symbols) and proliferation by CFSE dilution was determined 3 days later by measuring the number of CD4+ T cells (circles) and CD8+ T cells (squares) proliferating within each division. FIG. 8D and FIG. 8E: CFSE-treated human PBMCs were stimulated to proliferate with anti-CD3 and anti-CD28 antibodies (♦) in the presence of 50 μg/mL sCD83 (▪) or 100 ng/mL LPS+KDO (▴) and the number of CD4+ T cells (shown in FIG. 8D) or CD8+ T cells (shown in FIG. 8E) proliferating within each division was determined 6 days later. All data points were run as replicates and bars represent range of each individual data point. Data sets are representative of 2-5 experiments.

FIGS. 9A, 9B, 9C, 9D, 9E, and 9F: sCD83 modulates cytokine and PGE2 secretion and induces IDO activity. Culture supernatant was collected 24 hours after PBMCs were stimulated in the presence of sCD83 alone (open bars) or in combination with anti-IL-10 antibody or the COX2 inhibitor NS-398. Cytokine concentrations were determined by cytokine bead arrays for: IL-2 (FIG. 9A); IFN-γ (FIG. 9B); IL-6 (FIG. 9C); and IL-10 (FIG. 9D). PGE2 concentration was determined by using a competitive ELISA (FIG. 9E). (FIG. 9F) IDO activity was measured in 24 hour culture supernatant from PBMCs stimulated to proliferate with anti-CD3 and anti-CD28 antibodies. Some cultures were untreated (open bars) or treated with 50 μg/mL sCD83 (closed bars), in combination with 1 μM of the Cox2 inhibitor. Error bars represent standard deviation of cytokine bead array data sets. Data sets are representative of two experiments.

FIG. 10: Schematic model depicting the interaction of sCD83 protein with the TLR4/MD-2 complex. TLR4 pro-inflammatory signals originate after LPS is bound through the interaction with CD14 and MD-2. MD-2 binding to LPS induces a conformational change in the MD-2/TLR4 heterodimers allowing the transduction of signals through the IRAK family of tyrosine kinases. sCD83 binds directly to MD-2 within the MD-2/TLR4 complex, facilitated by CD44v6. The results of antibody-blocking experiments provide a role for the association of CD14 and CD44v6 within close proximity to the sCD83 binding site on the TLR4/MD-2 complex. sCD83 binding through TLR4/MD-2 results in a rapid and sustained loss of IRAK-1 leading to an altered signaling cascade towards an anti-inflammatory response.

### SUMMARY OF THE INVENTION

Antibodies are provided that mimic the biological activity of sCD83 by activating the TLR4 pathway in a manner that usually depends on the participation of sCD83 (herein referred to as the “sCD83 pathway”). In some embodiments, the antibodies are bispecific antibodies that bind to CD44 and to MD-2 so as to activate the sCD83 pathway. In some embodiments, the antibodies are bispecific antibodies that bind to CD14 and to MD-2 so as to activate the sCD83 pathway.

The antibodies provided have immunosuppressive activity and thus are useful for the treatment or prevention of an unwanted immune response in a subject, such as, for example, autoimmune disease, transplant rejection, and allergic reactions. Accordingly, pharmaceutical compositions and formulations comprising these antibodies are provided, as well as methods of using such compositions and formulations for the treatment of autoimmune diseases, transplant rejection, and allergic reactions.

In yet another aspect, the invention provides a method of improving transplantation outcome in a mammalian transplant recipient comprising administering to said recipient a therapeutically effective amount of an antibody of the invention. The antibodies of the invention may also be used with one or more other immunosuppressive agents, wherein said immunosuppressive agent acts synergistically with said antibody to treat or prevent an unwanted immune response in a subject, for example, to improve transplant outcome.

### DETAILED DESCRIPTION

Relationship Between TLR4 and sCD83

Bacterial lipopolysaccharides (LPSs) provoke an inflammatory response following their recognition by Toll-like Receptor 4 (TLR4), a recognition receptor of the innate immune system. The TLR4 co-receptor MD-2 functions to tether bacterial lipopolysaccharide (LPS) to TLR4 and, through heterodimerization of the receptor, leads to potent pro-inflammatory signals (Park et al. (2009) Nature 458: 1191-95). Ligand engagement of TLR4/MD-2 dimers induces a signaling cascade downstream of Myd88 activation involving the phosphorylation of the adaptor protein IRAK-1 via IRAK-4. Activated IRAK-1 tethers to TRAF6 and leads to the nuclear translocation of NF-κB, inducing a pro-inflammatory cytokine cascade (Ringwood and Li (2008) Cytokine 42: 1-7, Kollewe et al. (2004) J. Biol. Chem. 279: 5227-36, Kanakaraj et al. (1998) J. Exp. Med. 187: 2073-79).

The inventors provide herein for the first time direct evidence that sCD83, a known immunosuppressant, binds to the TLR4 co-receptor MD-2 with high affinity. The inventors show that binding of sCD83 to MD-2 alters the pro-inflammatory signaling cascade, resulting in rapid loss of IRAK-1 and leading to induction of the anti-inflammatory mediators IDO, IL-10, and PGE₂ in a COX-2 dependent manner. PGE₂ and IL-10 are anti-inflammatory mediators that block T-cell activation and proliferation and also shift T cell cytokine production from a Th1 to a Th2 profile. The inventors also show that sCD83 inhibits T cell proliferation, blocks 11-2 secretion, and renders T cells unresponsive to further downstream differentiation signals mediated by 11-2. Identification of MD-2 as the binding partner for sCD83 made it possible for the inventors to map signals delivered through sCD83 engagement of the TLR4/MD-2 complex that lead to this altered regulatory cascade.

CD83 is a molecule from the immunoglobulin superfamily of proteins (see, e.g., Zhou et al. (1999) J. Immunol. 149: 735-742; see also U.S. Pat. No. 7,169,898; for review, see Fujimoto and Tedder ((2006) J. Med. Dent. Sci. 53: 86-91). Human CD83, identified as a 45-kDa type 1 membrane glycoprotein, is found in vivo in both a membrane-bound (transmembrane) form and a soluble form (Fujimoto and Tedder (2006) J. Med. Dent. Sci. 53: 85-91; Hock et al. (2001) Int. Immunol. 13: 959-67). The membrane-bound form of CD83 is expressed on mature dendritic cells (DCs), B cells, macrophages, activated T cells and T regulatory cells (Prazma and Tedder (2008) Immunol. Lett. 115: 1-8; Kreiser et al. (2015) Immunobiology 220: 270-9), and its expression on DCs is involved in the activation of T-cell-mediated immune responses (Prechtel et al. (2007) J. Immunol. 178: 5454-64; Aerts-Toegaert et al. (2007) Eur. J. Immunol. 37: 686-95; Kruse et al. (2000) J. Exp. Med. 191: 1581-90; Kruse et al. (2000) J. Virol. 74: 7127-36). The soluble form of CD83 (“sCD83”) is a result of alternative splicing and shedding and is known to be a potent immune suppressor that inhibits T-cell proliferation in vitro, promotes allograft survival in vivo (e.g., Xu et al. (2007) Transpl. Int 20: 266-76), prevents corneal transplant rejection (e.g., Bock et al. (2013) J Immunol 191: 1965-75), and attenuates the progression and severity of autoimmune diseases and experimental colitis (e.g., Eckhardt et al. (2014) Mucosal. Immunol. 7: 1006-18). Prior to the instant application, sCD83 was known to bind to human peripheral blood mononuclear cells (PBMCs) and to human monocytes (Chen et al. (2011) Proc. Nat'l. Acad. Sci. USA 108: 18778-83), but the specific molecules to which sCD83 binds had not previously been identified, and the underlying mechanism by which sCD83 mediates its regulatory effects were unknown.

The mature CD83 polypeptide includes three structural domains: an extracellular Ig-like domain; a transmembrane domain; and a cytoplasmic domain. The human extracellular domain (“hCD83ext”), a form of sCD83, comprises a single Ig-like (V-type) domain which is encoded by at least two exons (see, e.g., Zhou et al. (1999) J. Immunol. 149: 735-742; GenBank ID #Z11697) and is expressed strongly on the cell surface of mature dendritic cells (“mDCs”). hCD83ext has been shown to inhibit DC-mediated T cell stimulation (Lechmann et al. (2001) J. Exp. Med. 194: 1813-1821) and is effective in treating the mouse model for multiple sclerosis, experimental autoimmune encephalomyelitis (“EAE”; see Zinser et al. (2004) J. Exp. Med. 200: 345-351). Other forms of sCD83 have also been shown to have immunosuppressive activity. For example, PCT publication WO 2009/142759 discloses an sCD83 comprising the extracellular domain of sCD83 in which the third cysteine of the extracellular domain of CD83 (amino acid residue 85) is an amino acid other than cysteine, and preferably is serine. This mutant, designated sCD83-m3, was shown to have immunosuppressive activity including delaying or preventing tissue damage and host mortality resulting from transplant rejection.

Efforts to investigate and develop sCD83 for therapeutic applications by Applicants and others have for many years been frustrated by a number of issues, including variability in the activity of sCD83 preparations, a lack of reliable assays for CD83 activity, and a lack of understanding of the molecular mechanism and pathway by which CD83 exerts its effects. Some assays for CD83 activity proved unreliable because they can be affected by contamination with substances other than CD83, such as the common contaminant LPS. Variability in the observed activity of CD83 preparations has caused controversy in the art for many years, leading some to believe that CD83 lacked efficacy (see, e.g., Pashine et al. (2008) Immunol. Lett. 115: 9-15 and the letter to the editor in the same publication disputing those findings, Zinser and Steinkasserer (2008) Immunol. Lett. 115: 18-19). Regarding the mechanism of action, some investigators had suggested that CD83 acts in a homotypic way (Bates et al. (2015) Mucosal Immunol. 8: 414-28), but that study did not demonstrate a clear biophysical interaction, and thus the identity of the sCD83 receptor remained unknown. The instant application provides data that confirms the binding of sCD83 to monocytes and extends the antigen presenting cell types binding sCD83 to include CD14⁺ plasmacytoid and myeloid dendritic cell subsets present in peripheral blood.

The inventors' work presented herein demonstrates that the tolerogenic properties of sCD83 depend on interactions with APCs via the TLR4/MD-2 complex to modulate early cytokine signaling pathways, thus promoting T cell unresponsiveness and active suppression of T cell activation. In sCD83-treated cultures, IL-2 and to a lesser extent IFN-γ are suppressed, and prior exposure to sCD83 induces a state of T cell anergy defined by a lack of subsequent proliferation when exposed to IL-2. Other experiments show that addition of sCD83 during T cell activation leads to blockade of T cell proliferation and altered cytokine secretion profiles, rendering T cells unresponsive to further downstream IL-2 expansion and in this manner inducing a form of IL-2 anergy in T cells. These data support the potential of sCD83 and antibodies with similar binding properties to have therapeutic efficacy in treating autoimmune and inflammatory diseases and alleviating transplant rejection.

### Novel Role for CD44v6

Data provided herein demonstrate a previously unknown role for CD44v6 in sCD83 cell surface binding. While the invention is not limited by any particular scientific theory, the data indicates that sCD83 acts to regulate the immune response by engaging the TLR4/MD-2/CD14-CD44v6 complex, resulting in a lack of IRAK-1 protein expression. This sustained loss of IRAK-1 results in the production of PGE₂ and IL-10 and increased IDO activity, all of which have the combined effect of inhibiting T cell activation.

CD44s or fragments thereof containing the standard extracellular domain (also referred to as “soluble CD44”) can be expressed on the cell surface in multiple forms depending on the expression of variant regions of the stem region (v1-v10) (Misra et al. (2015) Front. Immunol. 6: 201). While studying sCD83 binding to monocytes, the inventors discovered that some donor monocyte preparations that lacked MD-2 and the CD44v6 variant did not bind sCD83. However, monocytes could be stimulated with GM-CSF to upregulate CD44v6 protein expression, which restored sCD83 binding to the monocytes. MD-2 protein expression could be increased either by the addition of exogenous CD44 or by increasing endogenous expression, which also increased sCD83 cell surface binding.

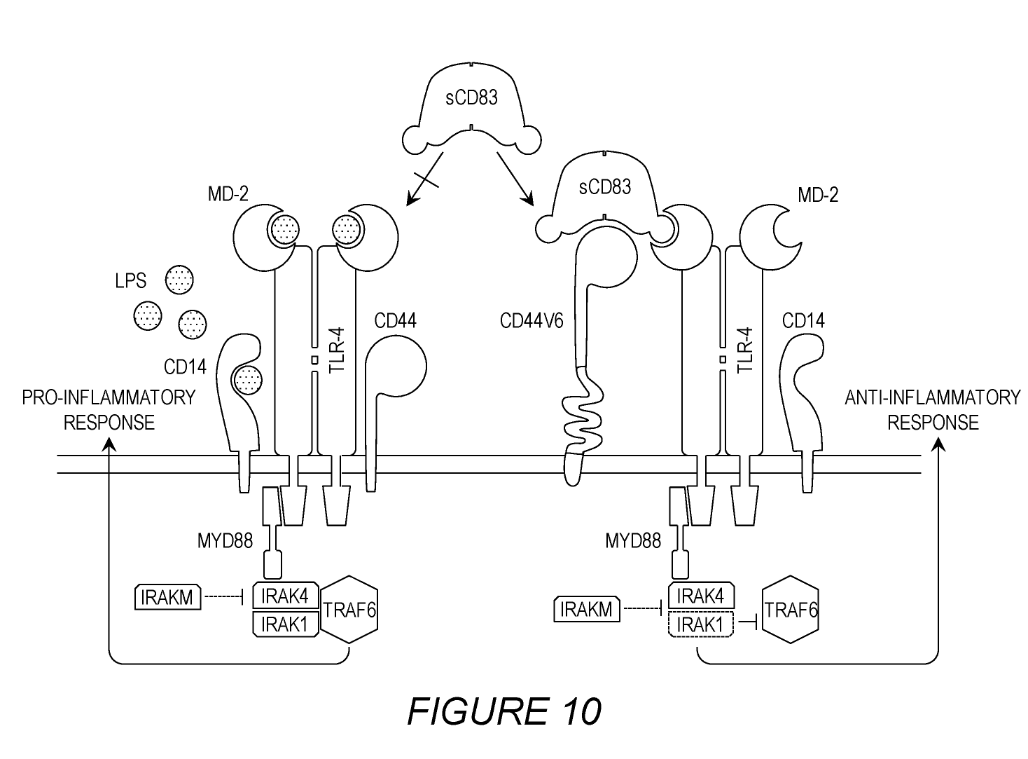

As shown by data presented in working Example 2, antibodies directed to the CD44s portion of the CD44 receptor blocked binding, but a direct protein-protein interaction between CD44s and sCD83 was not demonstrated. While the invention is not limited to a particular scientific mechanism, the data indicate that sCD83 binding to monocytes is dependent on the expression of the CD44v6 variant, which expresses the standard extracellular domain with at least the v6 variant of the stem loop region. The CD44v6 form of CD44 may facilitate the conformation necessary to allow sCD83 to bind to MD-2 within the complex (see schematic in FIG. 10).

Experiments described herein also show that the expression of CD14 and CD44 on monocytes provides necessary components of this unique complex of receptors and that the CD44 receptor v6 variant (in the stem loop region) expressed on monocytes is an accessory receptor associated with the sCD83-TLR4/MD-2 complex.

### Role of IRAK-1 and Downregulation of Immune Response

Regulation of TLR4 signaling occurs downstream at several check points to modulate excessive inflammation and especially during resolution of inflammation (Ringwood and Li (2008) Cytokine 42: 1-7; O'Neill (2008) Immunity 29: 12-20). TLR4 function has been investigated in models of autoimmune arthritis (Abdollahi-Roodsaz et al. (2007) Arthritis Rheum 56: 2957-67), myocardial ischemia (Shimamoto et al. (2006) Circulation 114: 1270-1274), and endotoxin-induced uveitis (Shen et al. (2014) Biochim. Biophys. Acta 1842: 1109-1120). Down regulation of IRAK-1 protein has been reported in models of LPS tolerance (Li et al. (2000) J Biol. Chem 275: 23340-45), and loss of IRAK-1 is reported in tolerogenic DCs (Albrecht et al. (2008) BMC. Immunol. 9: 69). Spontaneous models of colitis in mice using TLR4 knockouts revealed disease exacerbation which was dependent on TLR4 signals and IL-10 (Biswas et al. (2011) Eur. J. Immunol. 41: 182-94).

sCD83 has been shown to prevent EAE (Zinser et al. (2004) J. Exp. Med. 200: 345-51) and colitis (Eckhardt et al. (2014) Mucosal. Immunol. 7: 1006-18) in murine models, both of which have been shown to be critically dependent on IRAK-1 activity (Joh and Kim (2011) Br. J Pharmacol. 162: 1731-42, Deng et al. (2003) J. Immunol. 170: 2833-42). Prior studies (Lan et al. (2010) Transplantation 90: 1278-1285) showed that a short course of sCD83 administered to mice receiving orthotopic kidney allograft transplants resulted in graft survival of >100 days compared to a median survival in untreated animals of 35 days.

To test the mechanism of sCD83 action, the inventors devised a test for the dependency of sCD83 activity on TLR4 signaling using a transplant model. TLR4-deficient mice served as the donors for kidney transplants. Pilot experiments showed that even when recipient mice were treated with sCD83, the donor organ from TLR4-deficient animals was rejected in a similar time frame as those for recipient mice not treated with sCD83 (a median of 41.4 days). This suggests that, due to a lack of TLR4/MD-2 in the donor, the donor organ is no longer susceptible to the tolerogenic signals provided by sCD83 because there is no localized tolerance induced by sCD83-mediated secretion of IL-10 and PGE₂ at the organ site.

Antibodies that Affect the TLR4 Signaling Pathway

While the invention is not bound by a particular mechanism of operation, it is theorized that pro-inflammatory and anti-inflammatory signals from TLR4 result from the interactions diagrammed schematically in FIG. 10. In this scenario, the antibodies of the invention substitute for sCD83 in functionally connecting MD-2 and CD44 to produce an anti-inflammatory response via TLR4 and decrease the amount of IRAK-1 produced. In other embodiments, antibodies of the invention functionally connect MD-2 and CD14 to produce an anti-inflammatory response via TLR4. Thus, multispecific antibodies or bispecific antibodies of the invention bind to MD-2 and CD44 or to MD-2 and CD-14 to substitute for the molecular action of CD83 and produce anti-inflammatory signaling via TLR4. In other embodiments, multispecific antibodies or bispecific antibodies bind to MD-2 and CD11b or to CD44 and CD11b to substitute for the molecular action of CD83 and produce anti-inflammatory signaling via TLR4. In some embodiments, multispecific or bispecific antibodies bind to two or more molecules selected from the group comprising or consisting of MD-2, CD44, CD11b, and CD14 and produce anti-inflammatory signaling via TLR4.

Thus, in some embodiments, the invention provides a bispecific antibody comprising at least one anti-MD-2 antibody or antigen-binding fragment thereof and at least one anti-CD44 antibody or antigen-binding fragment thereof. In some embodiments, said at least one anti-MD-2 antibody or antigen-binding fragment thereof competes with sCD83-m3, LPS, or KDO for binding to MD-2. In some embodiments, said at least one anti-CD44 antibody or antigen-binding fragment thereof competes for CD44 binding with a reference antibody such as, for example, VFF-6, VFF-7, or VFF-18. In some embodiments, said at least one anti-CD44 antibody or antigen-binding fragment thereof does not compete for CD44 binding with a reference antibody such as, for example, IM7. In some embodiments, a bispecific antibody of the invention comprises at least one anti-CD44 antibody or antigen-binding fragment thereof that binds to the v6 region or the variant 6 region (see, e.g., Fox et al. (1994) Cancer Res. 54: 4539-46).

By “competes for binding” as used herein is intended that an antibody decreases the observed binding for another antibody. Techniques for measuring antibody binding and competition for binding are well known in the art. An antibody that competes for binding with another antibody or molecule or that blocks binding of another antibody or molecule decreases the binding observed for that antibody or molecule by at least 10%, 20%, 30%, 40%, 50%, 60%, 70%, or 100% or more.

### MD-2

MD-2 proteins, nucleic acid sequences, and structures are known in the art. The nucleic acid sequence of a human MD-2 cDNA is known in the art (GenBank Accession No. BAA78717.1) and also described, for example, in Shimazu et al. ((1999) J. Exp. Med. 189: 1777-82) and its structure discussed in detail, for example, in Park et al. (2009) Nature 458: 1191-95.

Thus, in some embodiments of a bispecific antibody of the invention, the antibody comprises an anti-MD-2 antibody or fragment thereof that binds to a portion of the MD-2 molecule in the region between Glu17 and Asn160, or that competes for binding to this region with the reference antibody 288307. The MD-2 antigen polypeptide recognized by the antibody may include additional residues of the full-length, native MD-2 protein which are outside the antigen domain.

### CD44

CD44 proteins, nucleic acid sequences, and structures are known in the art. The nucleic acid sequence of a human CD44 cDNA is known in the art (GenBank Accession No. M59040.1) and also described, for example, in Ham et al. (1991) Biochem. Biophys. Res. Commun. 178: 1127-34 and its structure discussed in detail, for example, in Zoller (2011) Nat. Rev. Cancer 11: 254-67.

In some embodiments, in a bispecific antibody of the invention comprising an anti-CD44 antibody or antigen-binding fragment thereof, the anti-CD44 or antigen-binding fragment binds to CD44 in the v6 region or the variant 6 region. Thus, in some embodiments of a bispecific antibody of the invention, the antibody comprises an anti-CD44 antibody or fragment thereof that binds to a portion of the CD44 molecule that comprises the extracellular 6 region, or that competes for binding to CD44 with the reference antibody VFF-6, VFF-7, or VFF-18. The sCD44 antigen polypeptide recognized by the antibody may include additional residues of the full-length, native CD44 protein which are outside the antigen domain.

### CD14

CD14 proteins, nucleic acid sequences, and structures are known in the art. The nucleic acid sequence of a human CD14 cDNA is known in the art (GenBank Accession No. CR457016) and also described and its crystal structure discussed in Kelley et al. (2013) J. Immunol. 190: 1304-11.

In some embodiments, in a bispecific antibody of the invention comprising an anti-CD14 antibody or antigen-binding fragment thereof, the anti-CD14 or antigen-binding fragment binds CD14 at or near the LPS binding site or competes for binding with the reference antibody My4 for binding to CD14.

### CD11b

CD11b proteins, nucleic acid sequences, and structures are known in the art. The nucleic acid sequence of a human CD11b cDNA is known in the art (GenBank Accession No. AH004143.2) and also described and its crystal structure discussed in Adair et al. (2013) PLoS ONE 8(2): e57951.

In some embodiments, in a bispecific antibody of the invention comprising an anti-CD11b antibody or antigen-binding fragment thereof, the anti-CD11b or antigen-binding fragment competes for binding with the reference antibody ICRF44 for binding to CD11b.

### Antibodies

By “bispecific antibody” is intended an antibody comprising two different binding specificities. In some embodiments, an antibody of the invention is a multispecific antibody comprising more than two different binding specificities. The production of antibodies having multiple binding specificities are known in the art (see, e.g., U.S. Pat. No. 9,416,197 (Goldenberg et al.; IBC Pharmaceuticals); Kontermann (2012) mAbs 4: 182-197; Jakob et al. (2013) mAbs 5: 358-363; U.S. Pat. No. 9,212,230 (Schuurman et al.; GenMab). In some embodiments, antibodies of the invention comprise at least one anti-MD-2 antibody binding region or antigen-binding fragment thereof and at least one anti-CD44 antibody binding region or antigen-binding fragment thereof. In some embodiments, antibodies of the invention comprise at least one anti-MD-2 antibody binding region or antigen-binding fragment thereof and at least one anti-CD14 antibody binding region or antigen-binding fragment thereof. In some embodiments, said antibody binding regions may comprise an entire antibody, so that the antibody of the invention comprises an anti-MD-2 antibody and an anti-CD44 antibody, or an anti-MD-2 antibody and an anti-CD14 antibody; so long as an antibody has the function of decreasing production of IRAK-1 leading to an altered signaling cascade towards an anti-inflammatory response, its use is contemplated in the methods of the invention.

The antibodies may be chimeric, humanized, or human antibodies. In some embodiments, the antibodies are humanized. For example, a humanized antibody of the invention can comprise the CDR sequences of, e.g., a murine anti-MD-2 antibody and the CDR sequences of, e.g., either a murine anti-CD44 or anti-CD14 antibody and the framework and constant region sequences from one or more human antibodies. Methods of antibody humanization are well known in the art. The antibody can be of various isotypes, including, e.g., IgG1, IgG2, IgG3, or IgG4. Numerous anti-MD-2, anti-CD14, and anti-CD44 antibodies are commercially available and/or known in the art and can be used to make the claimed compositions and/or to perform the claimed methods.

According to the invention, the term “antibody” includes antibody fragments of the invention that retain the binding properties specified. Techniques for making such modifications are well known in the art. An “antibody fragment” is a portion of an intact antibody such as F(ab′)2, F(ab)2, Fab′, Fab, Fv, sFv, scFv, dAb and the like. Regardless of structure, an antibody fragment of the invention binds with the same antigen recognized by the full-length antibody. For example, antibody fragments include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). “Single-chain antibodies” (scFv) consist of a polypeptide chain that comprises both a VH and VL domain which interact to form an antigen-binding site. Antibody fragments also include diabodies, triabodies, and single domain antibodies (dAb).

In other embodiments, the invention provides chimeric molecules comprising any of the herein described antibody regions fused to a heterologous polypeptide or amino acid sequence. Non-limiting examples of such chimeric molecules comprise any of the herein described antibody region fused, either at the N-terminus or C-terminus, to an amino acid sequence that imparts additional functionality, stability or homing properties. In some embodiments an antibody region is fused at the N- or C-terminus to an Ig or Fc domain of an immunoglobulin (e.g., IgG1, IgG2, IgG3, IgG4, IgA and IgGA2,), preferably a human immunoglobulin. Methods for making such molecules are known to those of skill in the art. The antibodies or fragments can be combined or joined in any suitable manner to produce an antibody of the invention. Antibodies of the invention can also be produced in vitro or synthesized and administered to a subject to produce therapeutic benefits described herein; thus, in some embodiments, an antibody of the invention is a synthetic antibody.

### Assays of Anti-Inflammatory Activity

In some embodiments, an antibody of the invention will bind to MD-2 and to CD44 and will have anti-inflammatory activity. In some embodiments, an antibody of the invention will bind to MD-2 and to CD14 and will have anti-inflammatory activity. Anti-inflammatory activity can be evaluated directly or indirectly and can be measured in vivo or in vitro; suitable assays are described herein in working Example 3 (the “IRAK assay”) and working Example 4 (the “Macrophage assay”). Both assays are relatively quick, reliable, in vitro assays that make testing antibodies of the invention for anti-inflammatory activity economically and practically feasible. Other assays are known in the art and can also be used to evaluate anti-inflammatory activity, as will be understood by one of skill in the art.

The IRAK Assay measures the rapid down-regulation of IRAK-1 protein by sCD83 in macrophage cultures. PBMCs were cultured in the presence of GM-CSF for 5 days. Macrophages were harvested and cultured overnight in media containing GM-CSF, then incubated in the presence or absence of a test antibody in 1 mL cultures for various times. Cell lysates were then used to prepare a Western blot and evaluated for IRAK expression as described in more detail in working Example 3.

The Macrophage Assay measures the polarization of pro-inflammatory macrophages (M1) to anti-inflammatory macrophages (M2). Mononuclear cells were isolated from leukapheresis of human patients and could be used immediately or stored frozen for later use. Cells were cultured in media supplemented with 1000 U/ml of GM-CSF for 5 days, after which pro-inflammatory (M1) macrophages were harvested and cultured for 3 or 4 days in the presence or absence of a test antibody. Macrophage differentiation was then assessed by flow cytometry using monoclonal antibodies conjugated with FITC, PE, or APC fluorochrome directed against various cell surface antigens. Pro-inflammatory generated macrophages (M1) are polarized to anti-inflammatory macrophage (M2) defined by phenotypic changes after exposure to an effective amount of an anti-inflammatory antibody.

Thus, antibodies of the invention can decrease the ability of mature immunostimulatory dendritic cells or other antigen-presenting cells to stimulate T-cell proliferation by at least 10%, 20%, 30%, 40%, 50%, 60%, 70%, 80%, or 90% or more in comparison to an appropriate control such as, for example, an interaction between mature immunostimulatory dendritic cells and T-cells in the absence of the antibody. The antibodies of the invention can be used in methods such as methods for decreasing the amount of IRAK-1 produced by a cell comprising exposing said cell to said antibody. In this manner, the invention provides methods for decreasing the amount of IRAK-1 produced by a cell, either in vitro or in a subject (i.e., in vivo) by exposing the cell to an antibody of the invention in vitro or by administering an antibody of the invention to a subject.

In some embodiments, an antibody or antibody fragment of the invention can decrease the expression of TNF-α, CD80 and/or CD83 by immunostimulatory dendritic cells matured in vitro in the presence of said antibody or antibody fragment during at least one step of the maturation process by at least 10%, 20%, 30%, 40%, 50%, 600/o, 70%, 80%, or 90% or more in comparison to an appropriate control (see, e.g., Lechmann et al. (2001) J. Exp. Med. 194: 1813-1821; WO 2004/046182). For example, the addition of an anti-inflammatory antibody to a culture comprising CD14+ monocytes or immature dendritic cells alters surface expression such that CD80 and CD83 expression is decreased after culturing for several hours or several days or by the mature DCs produced from such cells. The addition of a bispecific antibody of the invention to a culture comprising mature dendritic cells reduces CD83 expression, and DCs treated with the antibody lose their ability to stimulate T-cell proliferation. In this manner, the antibody can be said to have altered the phenotype or immunophenotype of the treated cells. The expression of cell surface markers of a population of cells can be evaluated by commonly-used techniques known to those of skill in the art such as, for example, FACS analysis, to determine the effectiveness of the antibody in methods of decreasing or increasing expression of one or more markers. Expression or production of other compounds by cells can also be monitored by methods known in the art to evaluate the effectiveness of an antibody of the invention in methods of altering cell phenotype.

An antibody of the invention is said to promote an anti-inflammatory response if it decreases the amount of IRAK-1 produced by PBMCs, monocytes, dendritic cells, or CD14+ PBMCs as measured by any suitable assay, for example, the IRAK assay presented in working Example 3. An antibody is said to have “anti-inflammatory” activity in such an assay if it decreases the amount of IRAK-1 produced by at least 10%, 20%, 300, 40%, 50%, 60%, 70%, 80%, 90% or more in comparison to an appropriate control or control antibody as measured in the same assay. Alternatively, an antibody is said to have “anti-inflammatory activity” if it increases the amount of IL-10 or PGE2 production by PBMCs, monocytes, dendritic cells, or CD14+ PBMCs by at least 10%, 20%, 30%, 40%, 50%, 60%, 70%, 80%, 90% or more in comparison to an appropriate control, such as, e.g., a control antibody.

Other assays of anti-inflammatory activity known in the art have been used to assess CD83 activity and so could also be used to assess an antibody of the invention by substituting said antibody for CD83 in an assay. Such assays include the ability of sCD83 to inhibit the production of TNF-α by LPS/IFN stimulated PBMCs, described in detail in Example 4 of PCT publication of WO 2009/142759 and referred to herein as the “TNF-α assay.” In this assay, the ability of sCD83 to inhibit TNFα production by LPS/IFN-γ stimulated peripheral blood mononuclear cells (PBMCs) was evaluated in an in vitro assay using PBMCs from cynomologus monkeys. Cynomologus monkey PBMCs were isolated and pretreated 12 hours with different concentrations of sCD83 (0.5, 5, 25 and 100 μg/ml). The cells were then activated for 6 hours with LPS (1 μg/ml) plus IFN-γ (100 U/ml) in the presence of Brefeldin A (4 μg/ml) and then intracellularly stained for TNFα using a commercially available Fix/Perm kit. Flow cytometry acquisition and analysis of the amount of TNFα production was evaluated using either myeloid dendritic cells or monocytes. The results were expressed as % of mDC or monocytes producing TNFα and in Mean Fluorescence Units (MFU), which represents the levels of TNFα on a per cell basis.

In some embodiments, anti-inflammatory activity can also be evaluated by the ability of an antibody to prevent rejection of a transplanted tissue, for example, using the mouse and rat kidney transplantation models for assessing allograft tolerance (as demonstrated in Examples 7 and 9 of WO 2009/142759). The mouse orthotopic kidney transplant model (see Zhang et al. (2005) Microsurgery 16(2): 103-109) can be used to assess the ability of an antibody to induce allograft tolerance as follows: BALB/c mice received kidney allografts from C57BU6 mouse donors; recipient mice are either untreated or treated with an anti-inflammatory agent one day prior to transplantation (day −1), the day of transplantation (day 0) and for seven post operative days (POD). Anti-inflammatory activity was demonstrated by a significant increase in length of post-operative survival time in treated mice.

The well-established rat renal transplant model (essentially as described in Bedard et al. (2006) Transplantation 81(6): 908-14) and U.S. Pat. No. 7,514,405) can also be used to assess anti-inflammatory activity. In this model, renal transplant recipients are treated with a short-term (11 days) dose of cyclosporine (CsA) to prevent initial acute rejection. Such transplant recipients reliably demonstrate that pathological changes characteristic of chronic graft rejection would occur without effective treatment by post-operative day (POD) 140. F344 rats served as renal donors to Lewis rats. Transplant recipients are sacrificed on POD 140 and transplanted kidneys are assessed for indications of chronic rejection by histology and immunohistochemistry. Renal histology was scored by an independent pathologist assessing tubular atrophy, glomerular atrophy, interstitial fibrosis, intimal thickness, cell infiltrates and cortical scarring on a scale of 0-4, wherein 0=normal, 1=minimal change, 2=mild change, 3=moderate change and 4=marked change. Effective treatments are identified as a significant improvement in scores in each category compared to a suitable control.

### Antibody Formulations

Antibody compositions can be processed together with suitable, pharmaceutically acceptable adjuvants and/or carriers to provide medicinal forms and medicaments suitable for the various indications and types of routes of administration. A suitable pharmaceutical composition can include carriers, any suitable physiological solution or dispersant or the like, solubilizers, adjuvants, stabilizers, preservatives, sustained release formulations, etc. The physiological solutions comprise any acceptable solution or dispersion media, such as saline or buffered saline. The carrier may also comprise antibacterial and antifungal agents, isotonic and adsorption delaying agents, and the like. Except insofar as any conventional media, carrier or agent is incompatible with the active ingredient, its use is contemplated. The carrier may further comprise one or more additional compounds, including but not limited to cytokines. Examples of such formulations and methods of their preparation are known in the art (see, for example, Remington's Pharmaceutical Sciences, 18th ed. (1985) Mack Pub. Co., Easton, Pa., U.S.).

Any route of administration may be used, including, but not limited to transcutaneous, intracutaneous (i.c.), intraperitoneal (i.p.), subcutaneous (s.c.), intramuscular (i.m.), intravenous (i.v.), internodal (i.n.), etc., for the delivery of antibodies of the invention to a subject. One of skill in the art will appreciate that different forms of administration will be suitable for different compounds and/or indications, and will be able to select the most appropriate method of administration. For example, psoriasis may be treated topically with a formulation suitable for administration to skin, while systemic lupus erythematosus may be treated by administration to the subject of a formulation suitable for intraperitoneal injection. Practitioners having skill in the art are familiar with criteria and methods for adjustment of dosages and administrations of compounds, such as, for example, assessment of results from conventional clinical and laboratory tests, including biochemical and immunological assays. Where appropriate, components of a medicament can be administered separately.

For therapeutic or prophylactic use, the antibodies of the present invention alone, or in combination with other immune modulatory compounds (e.g., tolerance-inducing antigens, Cyclosporin A, FK506 plus MMF, rapamycin plus CD45RB, corticosteroids, etc.), are administered to a subject, preferably a mammal, more preferably a human patient, for treatment or prevention in a manner appropriate for the medical indication.

### Therapeutic Uses of Antibodies of the Invention

The antibodies, antibody fragments, pharmaceutical compositions, and formulations disclosed herein are useful for the prevention, cure, reduction, and/or alleviation of at least one symptom of a disease or disorder caused by the dysfunction or undesired function of an immune response in a subject, such as, for example, multiple sclerosis, rejection of a transplanted tissue, type I diabetes, and HIV infection and/or AIDS.

The antibodies, antibody fragments, pharmaceutical compositions, and formulations of the invention may be used for the production of a medicament for the treatment or prevention of a disease or medical condition caused by the dysfunction or undesired function of an immune response, such as, for example, autoimmune diseases, allergies, asthma, rejection of a tissue transplant (such as an organ transplant), or an unwanted immune response to a therapeutic composition, such that the unwanted immune response is repressed. In this manner, the invention provides a medicament comprising an antibody of the invention for decreasing or suppressing an immune response in a subject; in some embodiments, said immune response is rejection of a transplanted tissue, type I diabetes, or another autoimmune response.

Thus, in some embodiments, the invention provides a method for treating or preventing at least one symptom of a disease or disorder caused by the dysfunction or undesired function of an immune response in a subject, comprising administering an antibody or antibody fragment, formulation, or medicament of the invention to said subject. In this manner, the invention provides methods for decreasing or suppressing an immune response comprising administering an antibody or antibody fragment, formulation, or medicament of the invention to said subject. Dysfunctions or undesired functions of an immune response that can be treated using the antibodies and methods of the invention include autoimmune diseases, transplant rejection, graft versus host disease and allergy. Autoimmune diseases that may be treated using the novel antibodies and antibody fragments of the invention include but are not limited to: systemic lupus erythematosus, autoimmune (Type I) diabetes, pemphigus vulgaris, Grave's disease, Hashimoto's thyroiditis, myasthenia gravis, automyocarditis, multiple sclerosis, rheumatoid arthritis, psoriasis, autoimmune uveoretinitis, vasculitis, a chronic inflammatory bowel disease such as Crohn's disease or ulcerative colitis, HLA B27-associated autoimmunopathies such as Morbus Bechterew, obstructive pulmonary disease (COPD), ankylosing spondylitis and AIDS/HIV infection. Other diseases and disorders that result from inflammatory responses may also be treated with the antibodies and formulations of the invention, including such diseases and disorders as heart disease or cardiovascular disease and arteriosclerosis, including atherosclerosis.

A subject is considered to be tolerized and/or to have been immunosuppressed (i.e., immune tolerance is considered to have been induced or acquired) if at least one symptom of a disease or disorder caused by the dysfunction or undesired function of an immune response involving dendritic cells, antigen presenting cells (including monocytes or macrophages), B cells, or T cells is prevented, cured, reduced, or alleviated in comparison to an untreated control or other appropriate control (e.g., in comparison to the symptom prior to treatment or to the expected severity of the symptom without treatment, if the treatment is intended to prevent the development of or reduce the severity of an immune response). In this manner, the invention provides methods for tolerizing a subject, suppressing an immune response, and/or inducing immunosuppression comprising administering an antibody of the invention to a subject. For example, in this manner, the invention provides a method of improving transplantation outcome. Those of skill in the art are familiar with the selection and application of methods of measurement and evaluation of symptoms as well as with the selection of appropriate controls. In some embodiments, the undesired immune response is directed toward a therapeutic composition, and an object of the invention is to tolerize the subject to such therapeutic composition (such as, for example, an antigen derived from a tissue transplant, or an AAV vector).

Thus, a subject is considered to be tolerized and/or immunosuppression or suppression of an immune response is considered to have occurred where at least one symptom of a disease or disorder caused by the dysfunction or undesired function of an immune response is reduced or alleviated by at least 10%, 20%, 30%, 40%, 50%, 60%, 70%, 80%, 90%, 95%, or 100% in comparison to an appropriate control. In embodiments where the treatment is intended to reduce the risk of a subject for developing an autoimmune disorder, that risk is reduced or alleviated by at least 10%, 20%, 30%, 40%, 50%, 60%, 70%, 80%, 90%, 95%, or 100% in comparison to an appropriate control; this assessment may be performed statistically on a population of subjects. In embodiments where the treatment is intended to tolerize a subject to a therapeutic composition, an undesired function of an immune response is reduced by at least 10%, 20%, 30%, 40%, 50%, 60%, 70%, 80%, 90%, 95%, or 100% in comparison to an appropriate control. In other embodiments, objects of the invention include the production of tolerogenic dendritic cells; in these embodiments, “symptom” refers to a parameter of behavior of the cells either in vivo or in vitro.

The methods of the invention are useful for therapeutic purposes and thus are intended to prevent, cure, or alleviate at least one symptom of a disease or disorder caused by the dysfunction or undesired function of an immune response. A symptom of a disease or disorder is considered to be reduced or alleviated if the symptom is decreased, increased, or improved, as appropriate, by at least 10%, 20%, 30%, 40%, 50%, 70%, 90% or more in comparison to an appropriate control, such as in comparison to the symptom prior to treatment or in comparison to the expected severity of the symptom where the treatment is intended to be preventive. One of skill in the art is familiar with techniques and criteria for evaluating changes in symptoms. Symptoms of diseases or disorders caused by the dysfunction or undesired function of an immune response are known to those in the art and include but are not limited to the following: abnormal histology of a transplanted tissue; abnormal function of a transplanted tissue; brief length of survival time following an event such as, for example, diagnosis or transplantation; abnormally or undesirably high or low level or number of indicator protein(s) or other compound(s) in the blood, such as undesired antibodies or undesired cells (e.g., antigen-specific dendritic cells or T cells); abnormally or undesirably high or low level or number of indicator cells in the blood or elsewhere in the body, e.g., an undesirably low level or number of regulatory T cells, so that an undesired immune response is initiated or maintained.

Where appropriate, in vivo tolerization or tolerance and/or immunosuppression may be measured using in vitro assays, such as, for example, in a mixed lymphocyte reaction using cells isolated from a subject. Similarly, tolerization or tolerance and/or immunosuppression achieved in cells in vitro may also be measured in in vitro assays using various types of cells, such as, for example, dendritic cells, T cells, or B cells. If tolerization or tolerance and/or immunosuppression is measured using an in vitro method, the tolerization or tolerance or suppression of an immune response is considered to have occurred if the response of the cells to an immune stimulus is decreased by at least 10%, 20%, 30%, 40%, 50%, 70%, 90% or more in comparison to an appropriate control. Suitable assays directly or indirectly measure immune response and are known in the art; they include, but are not limited to: mixed lymphocyte reaction assays; cytotoxicity assays; antibody titer assays; assays for the production of IL-10; assays for the production of TGF-β; evaluation of cell surface markers; assay for a change in IRAK-1 production; and macrophage assay (see working examples). Other measures of tolerization include increased survival time of transplant recipients and reduced damage to a tissue following transplantation.

The antibodies, antibody fragments, pharmaceutical compositions, and formulations of the invention may be co-administered with other immunosuppressive compounds. The term “immunosuppressive compound” refers to a compound which is capable of depleting the size of a population of T and/or B clones of lymphocytes or which is capable of suppressing their reactivity, expansion, or differentiation. Immunosuppressive compounds for use in the methods of the invention include, but are not limited to: calcineurin inhibitors, including cyclosporine (also known as “CsA,” marketed as Neoral® or Sandimmune®) and tacrolimus (also known as “FK506,” marketed as Prograf®); purine metabolism inhibitors such as mycophenolate mofetil (also known as “MMF,” marketed as Cellcept®) and azathioprine (marketed as Azasan® or Imuran®); proliferation inhibitors such as everolimus (marketed as Certican®) and sirolimus (also known as “rapamycin” or “Rapa,” marketed as Rapamune®); monoclonal antibodies (“mAb”), such as anti-CD45 and anti-CD45RB (see, e.g., U.S. Pat. No. 7,160,987); monoclonal antibodies directed against T-cells, such as OKT3; monoclonal antibodies directed against the IL-2 receptor, including humanized anti-TaT antibodies, such as basilixamab and daclizumab; substances which block T-cell co-stimulatory pathways, such as CTLA-4-Ig1 fusion protein; substances which are able to induce chimerism (i.e., the coexistence of donor and recipient immune cells, in which graft tissue is recognized as self); and non-myeloblative pre-transplantation treatments such as cyclophosphamide (marketed as Cytoxan®). For a discussion of immunosuppressives and their targets, see, e.g., Stepkowski ((2000) Expert Rev. Mol. Med. Jun. 21, 2000: 1-23).

By “effective amount” of a substance is intended that the amount is at least sufficient to achieve at least one object of the invention when administered to a subject according to the methods of the invention. Thus, for example, an “effective amount” of an antibody of the invention is at least sufficient to tolerize the subject to a therapeutic composition when it is coadministered to a subject with the antibody or to produce a measurable effect on at least one symptom of a disease or disorder caused by the dysfunction or undesired function of an immune response. The effective amount of an antibody may be determined with regard to symptoms exhibited by an individual subject or it may be determined from clinical studies or extrapolated from appropriate studies in model systems. Thus, for example, an effective amount of an antibody of the invention includes an amount that would be expected to produce a measurable effect on at least one symptom of a disease or disorder based on a dosage range determined in a clinical study utilizing a method of the invention. In some embodiments, an effective amount of an antibody of the invention is an amount that alters expression of one or more cell surface markers or cytokines in an in vitro assay; suitable assays are known in the art and further described herein.

An effective amount of an antibody of the invention can be administered in one or more administrations, applications or dosages. Suitable administrations, applications, and dosages will vary depending on a number of factors, including but not limited to: specific activity of the compositions; the formulation of the compositions; the body weight, age, health, disease and condition of the subject to be treated; and the route of administration of the compositions into the subject. One of skill in the art will readily be able to adjust the dosage and administration, etc., in order to achieve the best results. For example, antibodies or antibody fragments may be administered to a patient within a range having: a lower end of 0.01, 0.05, 0.1, 0.5, 1, 2, 5, 7, 10, 20, 50, 70, 100, 200, 500, or 700 mg/kg, or 1, 2, 5, 7, 10, 20, 50, or 100 g/kg; and an upper end of 0.05, 0.1, 0.5, 1, 2, 5, 7, 10, 20, 50, 70, 100, 200, 500, or 700 mg/kg, or 1, 2, 5, 7, 10, 20, 50, 100, or 200 g/kg.

Generally, as used herein, by “subject” is intended any animal in need of treatment. Thus, for example, a “subject” can be a human patient or a non-human mammalian patient or may be another patient that is an animal.

Where prevention of a disease or medical condition is desired, a subject may be treated at regular intervals (e.g., approximately every two years, every year, every six months, every two to four months, or every month). However, in all embodiments of the invention, the methods and compositions of the invention are administered to a subject that has been identified as having a particular disease or disorder caused by the dysfunction or undesired function of an immune response or to a subject that has been identified as being likely to develop a particular disease or disorder. For example, a subject may be identified as likely to develop such a particular disease or disorder as a result of examination of the subject's family history; of a medical test such as a genetic test or a test to determine the subject's enzyme or metabolite levels; or of being diagnosed with another disease or disorder. In this manner, for example, the methods of the invention may be used to treat an autoimmune disease, to prevent the development of an autoimmune disease, or to reduce the risk of a subject for developing an autoimmune disease. Generally, a course of treatment ends when the subject is no longer being treated to alleviate or prevent a particular disease or medical condition caused by the dysfunction or undesired function of an immune response. Thus, the invention provides a method of treatment or prevention of a disease or medical condition caused by the dysfunction, unwanted immune response or undesired function of an immune response, wherein an effective amount of an antibody of the invention is administered to a subject so that immunosuppression is achieved. In one embodiment, the unwanted immune response is selected from the group consisting of autoimmune diseases, transplant rejection and allergy.

In some embodiments, the methods of the invention provide the benefit of a synergistic effect produced by the coadministration of an antibody of the invention with at least one other immunosuppressive compound, such that the efficacy of the combined treatment is much higher than would be expected from combining the two individual treatments. Moreover, if an antibody is coadministered to a subject with two or more other immunosuppressive compounds, even greater benefits can be provided. In this manner, the invention provides combination (or “coadministered”) treatments which have greater efficacy than is provided by the individual treatments alone. In some embodiments, the combination or coadministered treatment provides greater benefit than the sum of the benefits provided by each individual treatment. In some embodiments, the combination or coadministered treatment provides a benefit that is greater than the benefit provided by individual treatment by at least 10%, 20%, 25%, 30%, 50%, 75%, 100%, 200%, or more, or by 1.5-fold, 2-fold, 3-fold, 4-fold, or more. In some embodiments, the combination or coadministered treatment provides a benefit that is greater than the sum of the benefits provided by each individual treatment by at least 10%, 20%, 25%, 30%, 50%, 75%, 100%, 200%, or more, or by 1.5-fold, 2-fold, 3-fold, 4-fold, or more.

In another aspect, a method of improving transplantation outcome in a mammalian transplant recipient is provided, comprising administering to said recipient a therapeutically effective amount of an antibody of the invention. The method can also include administering to said recipient one or more other immunosuppressive agents. In one embodiment, said immunosuppressive agent is Cyclosporin A or Cyclosporin G. In other embodiments, said immunosuppressive agents are rapamycin plus anti-CD45RB monoclonal antibody, or tacrolimus (FK506) plus mycophenolate mofetil (MMF).

In some embodiments, the methods of the invention comprising administration of an antibody of the invention to a subject are used to prevent, cure, or alleviate at least one symptom of rejection of a tissue transplant in a tissue recipient. Generally, treatment of transplant recipients according to the methods of the invention can include treatment prior to, in conjunction with (i.e., at the same time), and/or following the transplantation of the tissue. In some embodiments, the methods of the invention prevent, cure, or alleviate all adverse symptoms of rejection of a tissue transplant, such that continued treatment of the transplant subject to prevent rejection becomes unnecessary; in such embodiments, it is said that graft tolerance has been induced in the subject. Treatment of the transplant recipient with an antibody of the invention can reduce or prevent damage to the recipient and/or transplanted tissue. Such damage can result from, for example, ischemia reperfusion injury.

In some embodiments, a tissue to be transplanted is perfused with an antibody of the invention, or is exposed to or stored in a solution containing an antibody of the invention after removal from the tissue donor and prior to transplantation into the recipient; in this manner, the antibodies of the invention can be used to reduce or prevent damage to the tissue. In some embodiments, the tissue to be transplanted may be removed from the donor and then exposed to a solution containing an antibody of the invention; that is, for example, the tissue may be stored in and/or perfused with a solution containing the antibody. As a result, when this tissue is then introduced into the transplant recipient, the antibody in the tissue and on the surface of the tissue will be coadministered to the subject along with the tissue.

Thus, in some embodiments, the methods of the invention are used to prevent, cure, or alleviate at least one symptom of rejection of a tissue transplant in a tissue recipient. In such embodiments, the transplant recipient (“recipient” or subject) is treated with an antibody of the invention, which can be administered to the subject by any suitable method. Generally, treatment of transplant recipients according to the methods of the invention can include treatment prior to, in conjunction with (i.e., at the same time), and/or following the transplantation of the tissue. In some embodiments, the methods of the invention prevent, cure, or alleviate all adverse symptoms of rejection of a tissue transplant, such that continued treatment of the transplant subject to prevent rejection becomes unnecessary; in such embodiments, it is said that graft tolerance has been induced in the subject.

“Tissue” as used herein encompasses discrete organs and/or specialized tissues (e.g., liver, kidney, heart, lung, skin, pancreatic islets, etc.) as well as “liquid” tissues (e.g., blood, blood components such as plasma, cells such as dendritic cells, etc.); the term “tissue” also encompasses portions and subparts of discrete organs and “liquid” tissues. Those of skill in the art are familiar with methods of assessment and treatment of transplant recipients and donors in order to achieve the best possible outcome for both recipient and donor. Thus, those of skill in the art will readily be able to assess and adjust dosages and administration as appropriate for a particular subject.

### Definitions

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting of” shall mean excluding more than trace elements of other ingredients and substantial method steps for administering the compositions of this invention.

The term “dendritic cells (DCs)” refers to a diverse population of morphologically similar cell types found in a variety of lymphoid and non-lymphoid tissues (see, e.g., Steinman (1991) Ann. Rev. Immunol. 9:271-296). Dendritic cells constitute the most potent and preferred antigen presenting cells (“APCs”) in the organism, although other antigen presenting cells such as monocytes and macrophages may also be used with methods and compositions of the invention. While dendritic cells can be differentiated from monocytes, they possess distinct phenotypes. It has been shown that mature DCs can provide all the signals necessary for T cell activation and proliferation.

“Immune response” broadly refers to the antigen-specific responses of lymphocytes to foreign substances. Any substance that can elicit an immune response is said to be “immunogenic” and is referred to as an “immunogen.” All immunogens are antigens; however, not all antigens are immunogenic. An immune response of this invention can be humoral (via antibody activity) or cell-mediated (via T cell activation).

As used herein, “conservative amino acid substitution” refers to substitution of an amino acid for another amino acid within the same group of amino acids. Amino acids can be classified according to their R groups as follows: 1) nonpolar, aliphatic R groups; 2) polar, uncharged R groups; 3) aromatic R groups; 4) positively charged R groups; and 5) negatively charged R groups. Amino acids with nonpolar, aliphatic R groups include glycine, alanine, valine, leucine, isoleucine, and proline. Amino acids with polar, uncharged R groups include serine, threonine, cysteine, methionine, asparagine and glutamine. Amino acids with aromatic R groups include phenylalanine, and tyrosine. Amino acids with positively charged R groups include lysine, arginine and histidine. Amino acids with negatively charged R groups include aspartate and glutamate. Thus, for example, a conservative amino acid substitution could exchange a serine, threonine, cysteine, asparagine, or glutamine for a methionine.

The terms “polynucleotide,” “nucleic acid,” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-stranded, double-stranded, and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. In addition to a native nucleic acid molecule, a nucleic acid molecule of the present invention may also comprise modified nucleic acid molecules. As used herein, mRNA refers to an RNA that can be translated in a cell.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. Thus, “polypeptide” as used herein refers to peptide chains containing natural amino acids as well as synthetic amino acids and other non-naturally-occurring compounds.

“Gene delivery,” “gene transfer,” “transfection” and the like as used herein are terms referring to the introduction of an exogenous polynucleotide into a host cell regardless of the method used for the introduction. Transfection refers to delivery of any nucleic acid to the interior of a cell. Gene delivery refers to the delivery of a nucleic acid that may be integrated into the host cell's genome or that may replicate independently of the host cell genome. Gene delivery or gene transfer does not refer to introduction of an mRNA into a cell. Transfection methods include a variety of techniques such as electroporation, protein-based, lipid-based, and cationic ion based nucleic acid delivery complexes, viral vectors, “gene gun” delivery and various other techniques known to those of skill in the art. The introduced polynucleotide can be stably maintained in the host cell or may be transiently expressed. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are capable of mediating transfer of genes to mammalian cells, as is known in the art and described herein.

Vectors that contain both a promoter and a cloning site into which a polynucleotide can be operatively linked are known in the art. Such vectors are capable of transcribing RNA in vitro or in vivo, and are commercially available from sources such as Stratagene® Corp. (La Jolla, Calif.) and Promega® Corp. (Madison, Wis.). In order to optimize expression and/or in vitro transcription, it may be necessary to remove, add, or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, nucleic acids or proteins of this invention can be conjugated to antibodies or binding fragments thereof which bind cell surface antigens, e.g., TCR, CD3, or CD4.

It is said that a polynucleotide or polynucleotide region (or a polypeptide or polypeptide region) has a certain percentage of “sequence identity” to another sequence (for example, 80%, 85%, 90%, or 95%) when that percentage of bases (or amino acids) are the same in the two sequences when they are aligned. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. The percent identity between two amino acid sequences can determined using the Needleman and Wunsch ((1970) J. Mol. Biol. 48:444-453) algorithm as implemented in a computer program known in the art. The percent identity between two amino acid or nucleotide sequences can be determined using the algorithm of Meyers and Miller ((1989) CABIOS 4:11-17) which has been incorporated into several programs for this purpose; suitable programs are known in the art.

The term “isolated” means separated from constituents, cellular and otherwise, with which the polynucleotide, peptide, polypeptide, protein, antibody, or fragments thereof, are normally associated in nature. For example, with respect to a polynucleotide, an isolated polynucleotide is one that is separated from the 5′ and 3′ sequences with which it is normally associated in the chromosome. A mammalian cell such as a dendritic cell is isolated if it is removed from the anatomical site from which it is found in an organism. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated,” “separated,” or “diluted” polynucleotide, peptide, polypeptide, protein, antibody, or fragment(s) thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragment(s) thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as its glycosylation pattern.

“Host cell,” “target cell,” or “recipient cell” are intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The host cell, target cell, or recipient cell may be prokaryotic or eukaryotic, and suitable cells include but are not limited to bacterial cells, yeast cells, animal cells, and mammalian cells, e.g., murine, rat, simian or human.

A “subject” is a vertebrate, preferably a mammal, more preferably a human. Mammals that can benefit from application of the methods of the invention and treatment with antibodies of the invention include, but are not limited to, murines, simians, humans, farm animals, sport animals, and pets, including dogs and cats.

A “composition” is intended to mean a combination of active agent and another compound or composition, which can be inert (for example, a detectable agent or label) or have biological activity, such as an adjuvant. A “pharmaceutical composition” is intended to include the combination of an active agent with a carrier, inert or active, making the composition suitable for diagnostic or therapeutic use in vitro or in vivo.

As used herein, the term “pharmaceutically acceptable carrier” encompasses any known pharmaceutical carriers that are suitable for the purpose of administering an antibody of the invention and/or other compositions to a patient. Such pharmaceutical carriers are known in the art.

An “effective amount” is an amount sufficient to effect beneficial or desired results, such as suppressed immune response, treatment, prevention or amelioration of a medical condition (disease, infection, etc.). An effective amount can be administered in one or more administrations, applications or dosages. Suitable dosages will vary depending on body weight, age, health, disease or condition to be treated and route of administration.

In accordance with the above description, the following examples are intended to illustrate, but not limit, the various aspects of this invention. It is to be understood, although not always explicitly stated, that the reagents described herein are merely exemplary and that equivalents of such are known in the art.

Headings and subheadings are included herein solely for reference and do not limit or restrict the meaning of the terms and explanations thereunder.

### EXPERIMENTAL EXAMPLES

### General Methods:

For general techniques, see Current Protocols in Immunology, eds. Coico et al. (Wiley, Hoboken, N.J., 2016).

### Example 1—Determination of Mechanism of Action of sCD83

### Materials and Methods

### Preparation of Recombinant Soluble CD83 Protein

The extracellular domain of human CD83 (amino acids 20-145) was PCR-amplified and soluble CD83 (sCD83) was expressed in E. coli and purified as previously described (see Lechmann et al. (2002) Protein Expr. Purif 24: 445-52; Lechmann et al. (2001) J. Exp. Med 194: 1813-21, Xu et al. (2009) Protein Expr. Purif 65: 92-99). Briefly, sCD83 was purified using a 5 mL GSTrap™ column (Amersham® Pharmacia Biotech). The GST-sCD83 containing fractions were loaded onto an anion exchange column and proteins separated by three different linear salt gradients. The GST-sCD83 fusion protein was incubated with thrombin to separate the sCD83 protein from GST and loaded onto glutathione sepharose 4B columns. The flow-through containing recombinant human sCD83 protein was collected and a final gel filtration separation was performed loading the flow through onto a Superdex® 75 column.

### Soluble CD83 Binding to Peripheral Blood Monocytes and Dendritic Cells

Leukapheresis from healthy volunteers was provided by Key Biologics (Memphis, Tenn.). Mononuclear cells were isolated using Histopaque®-1077 (Sigma-Aldrich® Co.), re-suspended in FBS (Atlanta Biologicals, Inc.) containing 10% DMSO (Sigma-Aldrich® Co.), and stored in liquid nitrogen. Thawed PBMCs were washed twice in PBS (Cambrex™ Corp.) prior to use. sCD83 (2 μg) was incubated with 1×10⁶/100 μl of PBMCs re-suspended in BD® Stain Buffer (FBS) (BD® Biosciences) for 15 minutes at room temperature. In some instances, sCD83 protein was pre-incubated with recombinant MD-2 protein (1.5 μg) prior to addition to cells for binding. Goat anti-human CD83 biotinylated polyclonal antibody (R&D Systems®, Inc.) was used to detect sCD83 protein binding to viable monocytes.

For antibody blocking experiments, cells were pre-incubated with the indicated purified antibodies to CD14 (clone M5E2, BD® Biosciences; clone 61D3, eBioscience® Corp.; clone My4, Beckman Coulter®, Inc.), to CD44s (clone 515, BD® Biosciences; clone 1M7, eBioscience® Corp.; and clone 156-3c11 and clone 5F12, Invitrogen® Corp.), to CD44v6 (clone VFF-7, eBiosciences® Corp.) to TLR4/MD-2 (clone MTS510, eBioscience® Corp.) or to MD-2 (clone 984, eBioscience® Corp.; clone 288307, R&D Systems®, Inc.) for 15 minutes at room temperature without washing prior to addition of sCD83 protein. Isotype control IgG1 and IgG2a antibodies were purchased from eBioscience® Corp.. Cells were washed with 2 mL BD® Stain Buffer (FBS) and incubated with 5 μl of Goat anti-human CD83 biotin (R&D Systems®, Inc.) for 15 minutes at room temperature. Cells were washed twice with 2 ml BD® Stain Buffer (FBS) and incubated with 20 μl of Streptavidin APC (BD® Biosciences) for 15 minutes at room temperature. Background staining was determined without the addition of sCD83 protein. Cells were washed twice, centrifuged, and re-suspended in BD® Stain Buffer (FBS) containing 7-AAD (BD® Biosciences) and signal was acquired with a FACSCalibur™ Flow Cytometer BD® Biosciences). sCD83 protein was directly labeled using Alexa Fluor® 488 Protein Labeling Kit (Invitrogen® Corp.) according to the manufacturer's instructions for competition binding experiments. Mouse anti-CD14 FITC (BD® Bioscience) was used to identify monocyte surface CD14 while donkey anti Mouse IgG PE (eBioscience® Corp.) was used to indirectly detect surface TLR4 and MD2 after binding with sCD83. Detection of sCD83 binding to dendritic cells was performed using the indirect method described in conjunction with anti-CD303 FITC, anti CD1c PE (Miltenyi Biotec), anti CD2 Qdot 605, anti CD14 Qdot 655 (Invitrogen® Corp.), and anti PE-Cy7 CD123 (BD® Biosciences) antibodies. Cells were washed, centrifuged and re-suspended in BD® Stain Buffer (FBS) containing 7-AAD and acquired with an LSRII Flow Cytometer (BD® Biosciences). Analysis was performed using Flowjo software ver.9.7 (Tree Star, Inc.)

Generation of mRNA for Monocyte Electroporation

Plasmids encoding CD14, MD-2, and TLR4 were purchased from OriGene® Technologies, Inc. The CD14 sequence used was Homo sapiens CD14 molecule transcript variant 1 (Accession Number NM_000591.1), and the MD-2 sequence used was Homo sapiens lymphocyte antigen 96 (LY96) (Accession Number NM_015364.2). The CD14 and MD-2 plasmids were digested with restriction enzyme XbaI and size fractionated by agarose electrophoresis; the 1500 bp band was then purified from the gel with a QIAquick Gel Extraction kit (Qiagen® Inc.) according to the manufacturer's protocol. Two μg of DNA fragment were used as the template in an in vitro transcription reaction using the Ampliscribe T-7 Flash IVT kit (Epicentre® Inc., Madison, Wis.). The RNA was purified over RNeasy Mini columns, polyadenylated, and capped as previously described (Slagter-Jager et al. (2013) Mol. Ther. Nucleic Acids 2: e91). RNA was purified over RNeasy MinElute columns (Qiagen® Inc.) to yield capped and polyadenylated RNA.

The TLR4 plasmid encoding Homo sapiens toll-like receptor 4 (TLR4), transcript variant 1 (Accession Number NM_138554.2) was purchased from OriGene® Technologies, Inc.. The pCMV6-XL4 plasmid was linearized with restriction enzyme SmaI, and the linearized plasmid was used as a template in a PCR reaction. The primers used were TLR4 reverse primer (5′-gcataaggcctgacatgtggcagc-3′) and T3-TLR4 forward primer, which has the sequence 5′-GCAATTAACCCTCACTAAAGGActgctgctcacagaagcagtgaggaCgaCgccagg-3′ where theT3 promoter is shown capital letters and the C-residues in bold are T to C substitutions to remove internal ATG codons. The PCR reaction buffer contained 10× Platinum Taq PCR buffer (Invitrogen® Corp.), 10 mM each dNTPs (Clontech® Laboratories, Inc.), 50 mM MgCl₂, primers (5 mM, IDT), linearized plasmid, Platinum Taq DNA polymerase (Invitrogen® Corp.) and water. The reaction was performed in a GeneAmp PCR System (Applied Biosystems®, LLC) with the following program: 94° C., 2 min; 35 cycles of 94° C., 30 sec; 55° C., 30 sec; 72° C., 2 min. The 2.5 kb fragment was purified with a QIAquick Gel Extraction kit (Qiagen®, Inc.) according to manufacturer's instructions and DNA was sequenced to confirm the two sequence substitutions (ACGT, Wheeling, Ill.). For in vitro transcription (IVT), PCR fragments were used as templates in an IVT reaction, using Ampliscribe T3 High Yield Transcription kit (Epicentre® Inc., Madison, Wis.) according to the manufacturer's instructions. RNA was purified over RNeasy Mini columns, and an aliquot of RNA was taken further to a capping reaction and subsequently to a polyadenylation reaction as described (53). RNA was purified over 4 RNeasy MinElute columns (Qiagen®, Inc.) according to the manufacturer's instructions to yield capped and polyadenylated TLR4 RNA. PBMCs were electroporated with RNA encoding CD14, MD-2, or TLR4 and assayed for soluble CD83 binding using the indirect method described above.

### Cell Free Binding Assay

sCD83 was prepared at 1 μg/mL in Coating Buffer (BD® Bioscience OptEIA ELISA Reagent Set B) and incubated overnight at 4° C. Plates were washed with wash buffer (PBS with 0.05% Tween 20, BD® Bioscience OptEIA Reagent Set B) three times using an ELx405 auto plate washer and then 300 μl of blocking buffer (1% BSA in PBS) was added to each well. The plates were then incubated for 2 hours at 37° C. to block and then washed three times with wash buffer. The protein of interest (MD-2, CD14, MD-2+LPS, or LPS) (R&D Systems®, Inc., Sigma) was serially diluted in seven tubes, and then 100 μl of the diluted protein samples were transferred to the appropriate wells on the capture plate. The plates were then incubated for 1 hour at room temperature in the dark. Plates were washed and goat anti-human biotinylated CD83 detection antibody (R&D Systems®, Inc.) was added at 100 μl/well and incubated for 2 hours at room temperature in the dark. The plates were then washed, and streptavidin-HRP was prepared at a 1:1000 dilution and added at 100 μl/well. The plates were incubated for 1 hour at room temperature in the dark and then washed, and development solution was prepared by combining equal parts of Substrate A plus Substrate B (BD® OptEIA ELISA Reagent Set B) and added to the plates (100 μl/well). The plates were allowed to develop for 1-10 minutes and the reaction was stopped by adding 50 μl/well of stop solution (BD® OptEIA reagents set B). The plates were analyzed on a ELx800 universal microplate reader at 450 nm.

### Western Blotting

Monocytes were isolated from PBMC using negative selection (Stemcell) and cultured in R-10 medium ((10% fetal bovine serum (FBS; Atlanta Biologicals), Roswell Park Memorial Institute-1640 (supplemented with 10 mM 4-2-hydroxyethyl-1-piperazineethanesulfonic acid pH 7.4, 1 mM sodium pyruvate, 0.1 mM nonessential amino acids, 2 mM sodium glutamate, and 55 mM 2-mercaptoethanol; Invitrogen® Corp.)) with GM-CSF overnight. sCD83 or LPS was then added for the indicated time points. Cells were lysed in ice cold RIPA buffer in the presence of a protease/phosphatase inhibitor cocktail (Pierce ECL Plus Western Blotting Substrate). Bradford (BCA) protein assay (Thermo Scientific) was performed to determine total protein concentrations. 4-5 μg of each lysate sample was run on each lane of a Criterion 10-20% Tris-HCL gel at 200V for 50 minutes. The gel was transferred to PVDF membrane at 100V for 75 minutes. The PVDF membrane was washed in Tris-Buffered Saline with 0.01% Tween 20 (TBS-T), blocked in 5% nonfat milk in TBS-T for 1 hour, and washed 3× in TBS-T. The washed membrane was incubated with primary antibody (1:1000): Anti IRAK-1, IRAK-2, IRAK-M, IRAK-4 and TRAF6 antibodies (Cell Signaling) in 3% BSA in TBS-T at 4° C. overnight. The membrane was washed, then incubated with anti-rabbit HRP (1:2000) in blocking buffer for 1 hr. The blot was then developed using ECL Plus kit.

### T Cell Stimulation Assay

Human PBMCs from normal donors were thawed overnight in R-10 medium. Cells were labeled with CFSE using the CellTrace CFSE Cell Proliferation Kit for Flow Cytometry (Life Technologies). CFSE labeled cells were incubated with varying concentrations of sCD83 protein. To stimulate proliferation of PBMCs, anti-human functional grade purified CD3 and CD28 antibodies (eBioscience® Corp.) were added to the wells at a concentration of 0.123 ng/mL, and cells were incubated for 6 days at 37° C. On day 6, cells were harvested and stained with antibodies against CD3 (PECy7, BD® Biosciences), CD4 (Pacific Blue, BD® Biosciences), and CD8 (PE-Texas Red-X (ECD), Beckman Coulter®, Inc.) for 15 minutes at room temperature. LIVE/DEAD Fixable Aqua Dead Cell Stain Kit (Life Technologies) was used as a viability marker. Cells were washed twice with FACS stain buffer (with FBS; BD® Biosciences), stained with the Aqua Dead Cell Stain Kit for 15 minutes at room temperature, and washed twice more with the FACS stain buffer. Fluorescence was measured on an LSRII (BD® Biosciences) flow cytometer, and a total of 100,000 events were collected for each sample. Cell proliferation was measured by the distribution of CFSE in each sample. Fluorescence was measured on an LSRII flow cytometer using FACSDiva software (BD® Biosciences). Data were analyzed with FlowJo software (Tree Star).

### Detection of Cytokine Secretion

To evaluate cytokine secretion from stimulated PBMC cultures in response to sCD83 treatment, the supernatant was collected after culture and analyzed for human cytokines using the Cytometric Bead Array kit (BD® Biosciences) according to the manufacturer's instructions. Data were analyzed using the FCAP Array software (BD® Biosciences). PGE₂ concentrations were measured using a competitive ELISA kit (Thermofisher).

Determination of IDO activity.

IDO activity in PBMC cultures was assessed by detecting tryptophan and the tryptophan catabolite kynurenine, which is generated by cells expressing IDO. Kynurenine and tryptophan in culture media were measured by HPLC after deproteination using a C18 reverse phase column as described (Huang et al. (2012) J. Immunol. 188: 4913-4920; Laich et al. (2002) Clin. Chem 48: 579-581). Data are expressed as the ratio of kynurenine:tryptophan (K:T).

### Results

### Soluble CD83 Binds to CD14⁺ Monocytes Expressing the TLR4 Co-Receptor MD-2

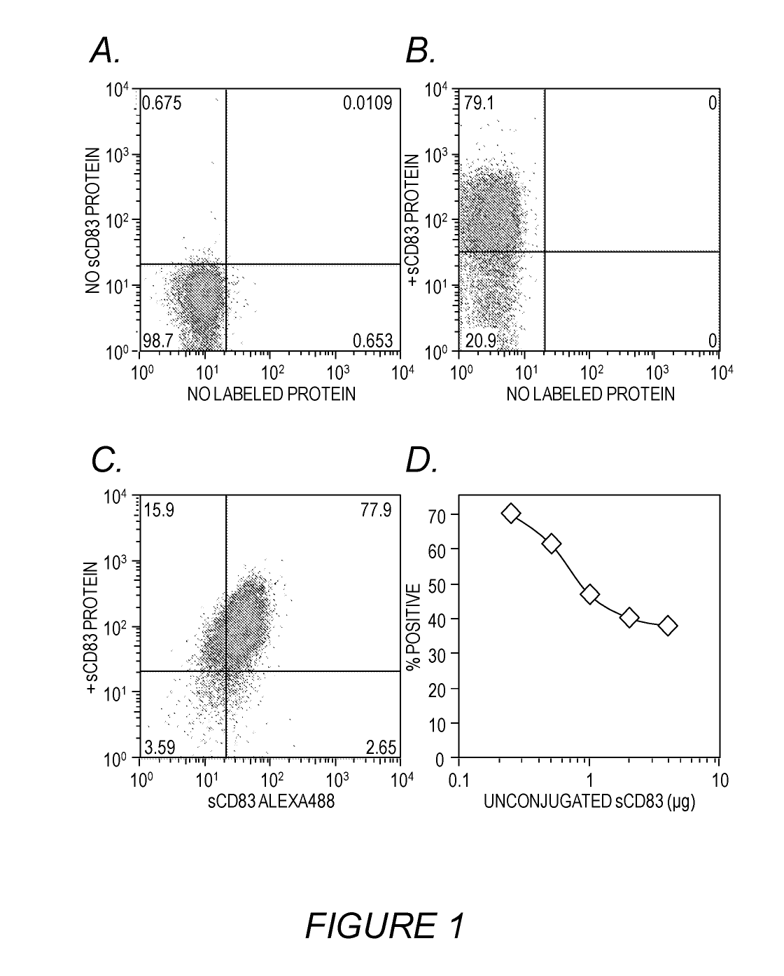

PBMCs were incubated with sCD83 protein and cell surface binding was detected on CD14′ monocytes using a biotinylated anti-CD83 specific antibody (FIG. 1B). Monocytes do not express membrane bound CD83 and do not show positive staining when the secondary anti-CD83 antibody is added to cells in the absence of sCD83 protein (FIG. 1A). To show direct cell binding of sCD83 protein, sCD83 protein was directly labeled with Alexa488 dye. Flow cytometry analysis revealed that both fluorescent labeled protein and unlabeled sCD83 protein detected with the indirect biotinylated anti-CD83 antibody bound to the same cells, resulting in 77.9% of the monocytes double stained with both reagents (FIG. 1C).

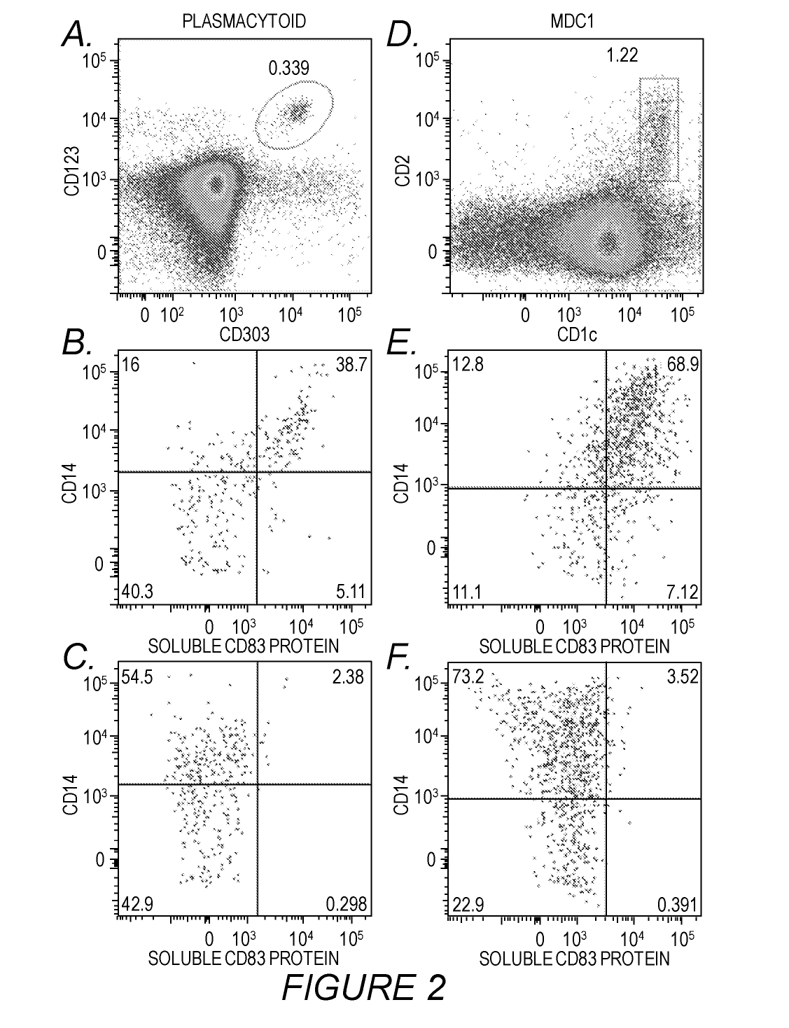

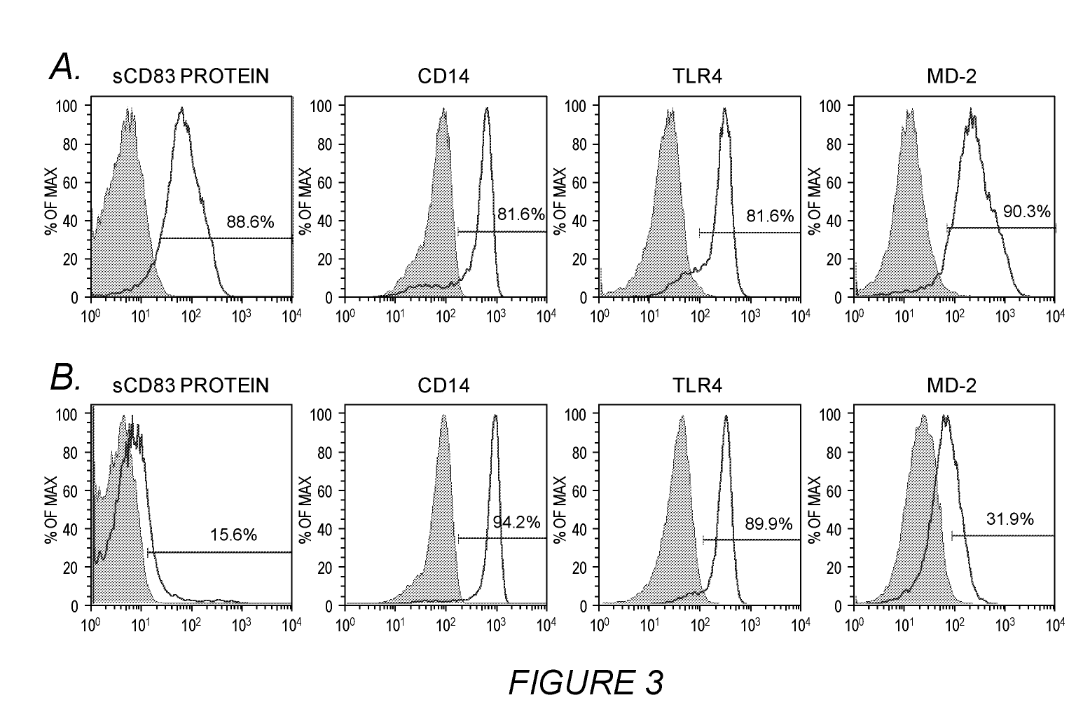

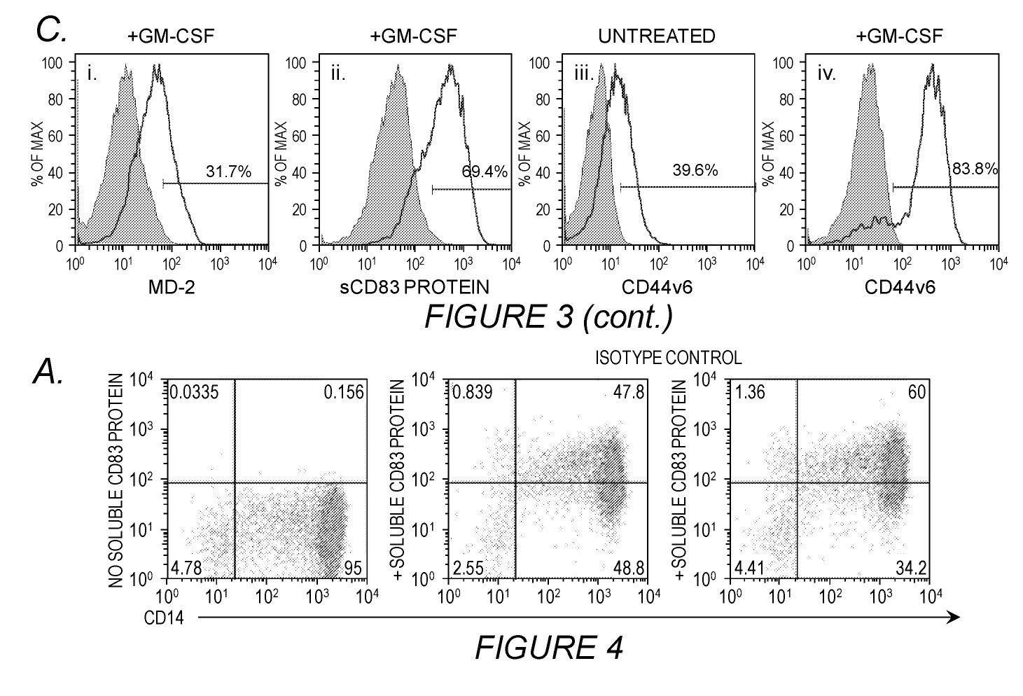

To confirm specificity of sCD83 binding to monocytes, a competition binding assay was performed. Direct binding of Alexa488-labeled sCD83 was inhibited when titered concentrations of unlabeled sCD83 protein were pre-incubated with the cells, demonstrating sCD83 specific binding to the cell surface of monocytes (FIG. 1D). The binding of sCD83 to other antigen presenting cells present in PBMCs was then investigated. Mature DCs present in the peripheral blood were identified by the expression of CD123 and CD303 for plasmacytoid DCs (Ziegler-Heitbrock et al. (2010) Blood 116: e74-e80; FIG. 2A) or by the expression of CD1c and CD2 for monocyte-derived DCs (FIG. 2D). sCD83 binding could be detected on a subset of CD14⁺ DCs within both the mature plasmacytoid (FIG. 2B) and myeloid DC subsets (FIG. 2E). Neither population of DCs expressed surface CD83, as evidenced by a lack of staining with the biotinylated anti-CD83 antibody (FIG. 2C, 2F). Monocytes binding sCD83 protein are positive for cell surface expression of CD14, TLR4 and MD-2 (FIG. 3A). Further binding analysis using a second donor which bound low levels of sCD83 (15.6%) revealed similar percentages of CD14 and TLR4 compared to donor 1, but a lower percentage of MD-2 (31.9% compared to 90.3% (FIG. 3B)). Monocytes from donor 2 were then treated with GM-CSF to see if activation would upregulate MD-2 and restore sCD83 binding. Culturing donor 2 monocytes induced little change in MD-2 expression (FIG. 3C(i)), but did increase sCD83 binding from 15.6% (FIG. 3B) to 69.4% (FIG. 3C(ii)). Interestingly, a concurrent increase in CD44v6 expression from 39.6% (untreated FIG. C(iii)) to 83.8% after GM-CSF stimulation (FIG. 3C(iv)) was detected.

CD44 Receptor Containing the v6 Variant is Essential for sCD83 Binding

The data obtained from these experiments showed that sCD83 binding cells are CD14, MD-2, TLR4 and CD44v6 positive, all molecules that associate with the TLR4 heterodimer (da Silva et al. (2001) J. Biol. Chem. 276: 21129-35; Taylor et al. (2007) J. Biol. Chem. 282: 18265-75). Antibody blocking experiments were then used to determine which molecules in the TLR4/MD-2/CD14-CD44 complex were necessary or essential for the binding of sCD83 to the surface of monocytes.

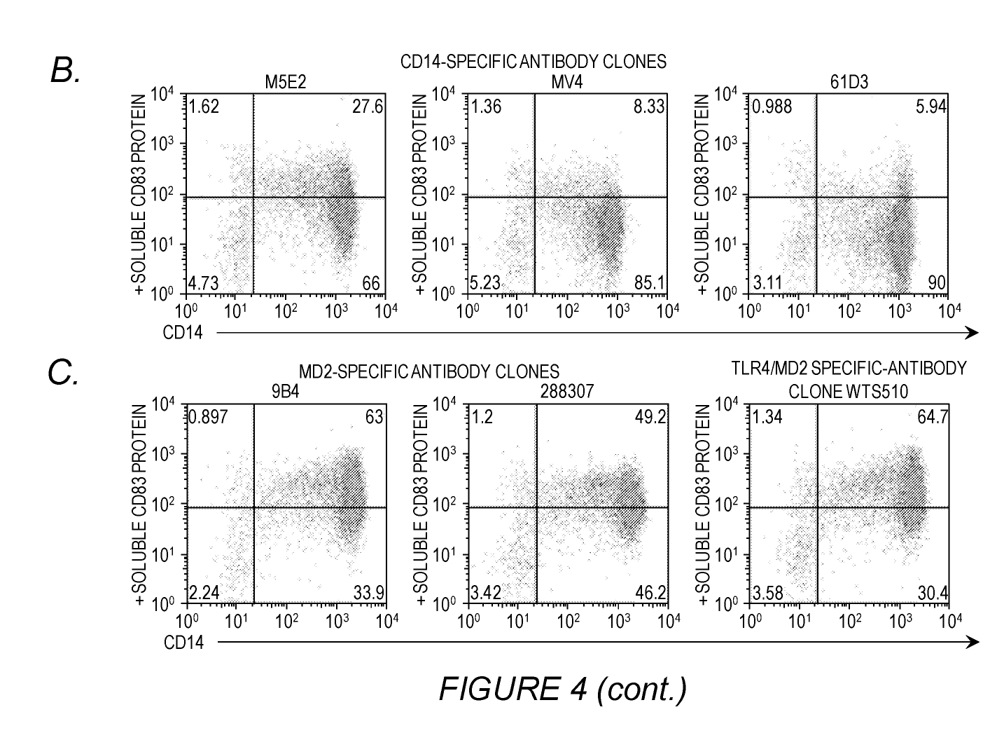

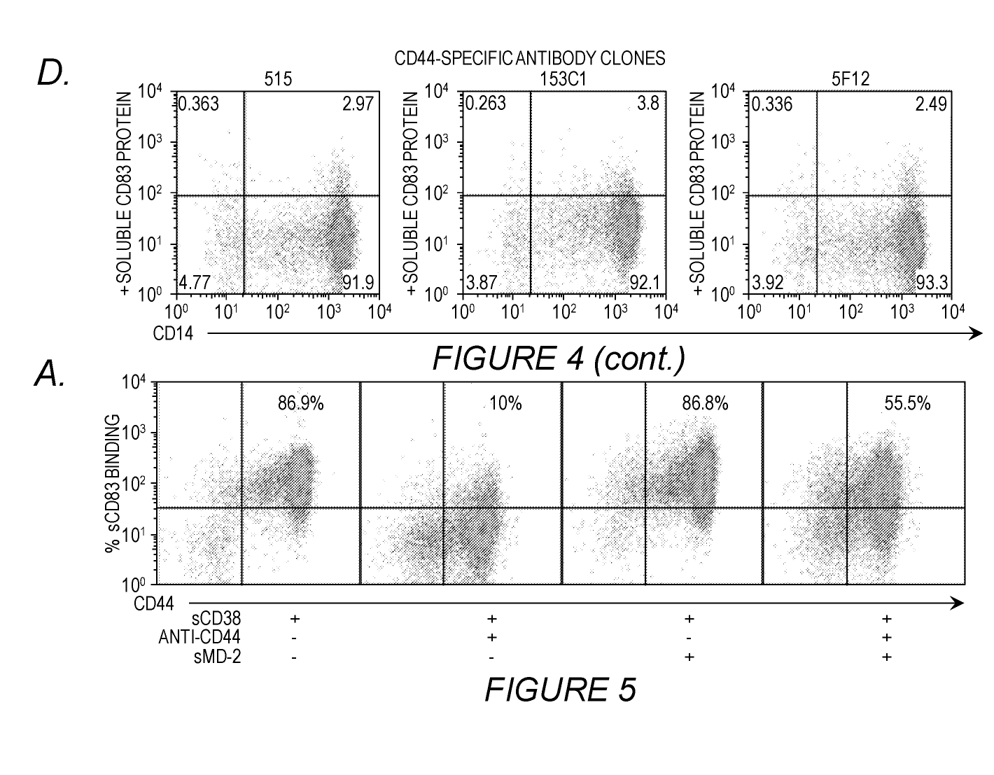

These experiments showed that sCD83 bound to 47.8% and 60% of CD14⁺ monocytes in the presence of either isotype control antibody (FIG. 4A). Pre-incubation of monocytes with anti-CD14 antibody clone M5E2 reduced sCD83 binding to 27.6% and clones My4 and 61D3 reduced sCD83 binding to 8.3% and 5.9%, respectively. All three of these antibody clones can detect surface expression of CD14, but clone M5E2 is reported not to be able to block LPS-induced activation of monocytes (Vanlandschoot et al. (2005) J Gen. Virol 86: 323-31). Because M5E2 blocked less sCD83 binding than the other clones, these data indicate that sCD83 interacts with a region associated with LPS binding.

The ability of anti-MD-2 antibodies to block sCD83 surface binding was then evaluated (FIG. 4C). Only one of the two MD-2 specific antibodies tested (clone 288307) partially blocked sCD83 binding to 49.2% versus 63% for clone 9B4. Furthermore, the antibody clone MTS510 with specificity for the complexed TLR4/MD-2 molecules did not block CD83 binding (64.7%).

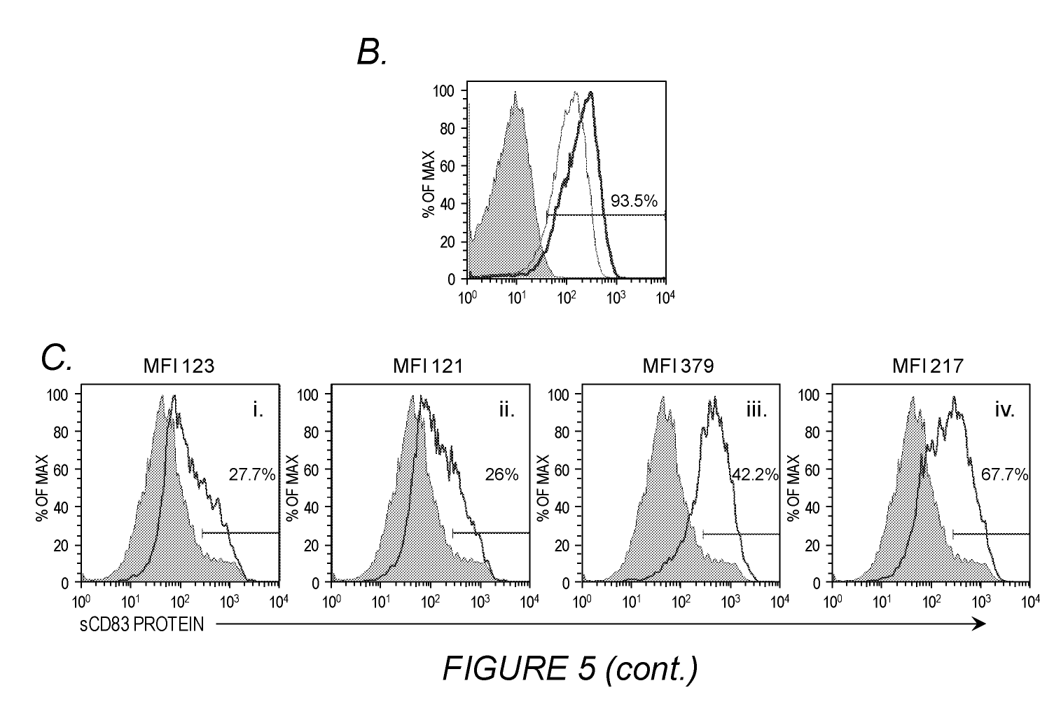

We then evaluated the contribution of the CD44 receptor to the ability of cells to bind sCD83, particularly focusing on the CD44v6 region. To do this, we studied sCD83 binding in the presence of antibodies that target the standard extracellular region of CD44. All three anti-CD44 antibody clones with specificity for the standard extracellular region of CD44 completely blocked sCD83 binding to the surface of monocytes, with residual sCD83 binding at 3-5% (FIG. 4D). However, if sCD83 protein was pre-mixed with soluble recombinant MD-2 protein prior to addition to cells, cell surface binding was restored to 55.5% from 10% in the presence of anti-CD44 blocking antibody (FIG. 5A). If the donor monocytes express CD44v6, antibody with specificity for CD44v6 region which recognizes an epitope along the stem region of the receptor does not block sCD83 binding (FIG. 5B). Since all CD44 receptors express the standard extracellular region but vary in the variant regions present in the stem loop, we conclude that the extracellular domain of CD44 is necessary for sCD83 binding, and expression of at least the variant 6 region is required to facilitate sCD83 binding. Furthermore, increased abundance of MD-2 protein can overcome anti-CD44 antibody inhibition of sCD83 binding.

Upregulation of MD-2 Enhances sCD83 Binding

The sCD83 cell-surface-binding experiments revealed variations in the binding of sCD83 to monocytes immediately post thaw for certain donor PBMC collections. For example, PBMCs from Donor 2 lacked surface expression of MD-2, which coincided with a lack of sCD83 binding (FIG. 3B). We then investigated the role of MD-2 expression on the cell surface of monocytes in facilitating sCD83 binding. The TLR4/MD-2/CD14/complex was hyper expressed on PBMCs by electroporating PBMCs with RNA encoding CD14, MD-2, or TLR4 and assayed for sCD83 binding to the surface of monocytes. Mock-electroporated monocytes and monocytes electroporated with CD14 RNA showed little difference in sCD83 binding, with percentages of 27.7% FIG. 5C(i)) and 26% (FIG. 5C(ii)), respectively. TLR4- and MD-2-electroporated monocytes showed increased binding of 42.2% (FIG. 5C(iii)) and 67.7% (FIG. 5C(iv)), respectively. Additionally, flow cytometry data showed an increase in the MFI (Mean Fluorescence Intensity) of sCD83 binding when monocytes were electroporated with MD-2 (MFI=217) or TLR4 (MFI=379) RNA compared to either monocytes electroporated with CD14 RNA (MFI=121) or mock electroporated (MFI=123), indicating that enhanced expression of MD-2 protein on the cell surface facilitates sCD83 binding.

sCD83 Binds to MD-2 with High Affinity

In the experiments described above, the inventors showed that the binding of sCD83 to monocytes was blocked with antibodies to CD14 or CD44 and that MD-2 can mediate CD44-independent binding of sCD83 to the cell surface of monocytes. The inventors then sought to determine the binding affinities between sCD83 and the co-receptors CD14, CD44 and MD-2.

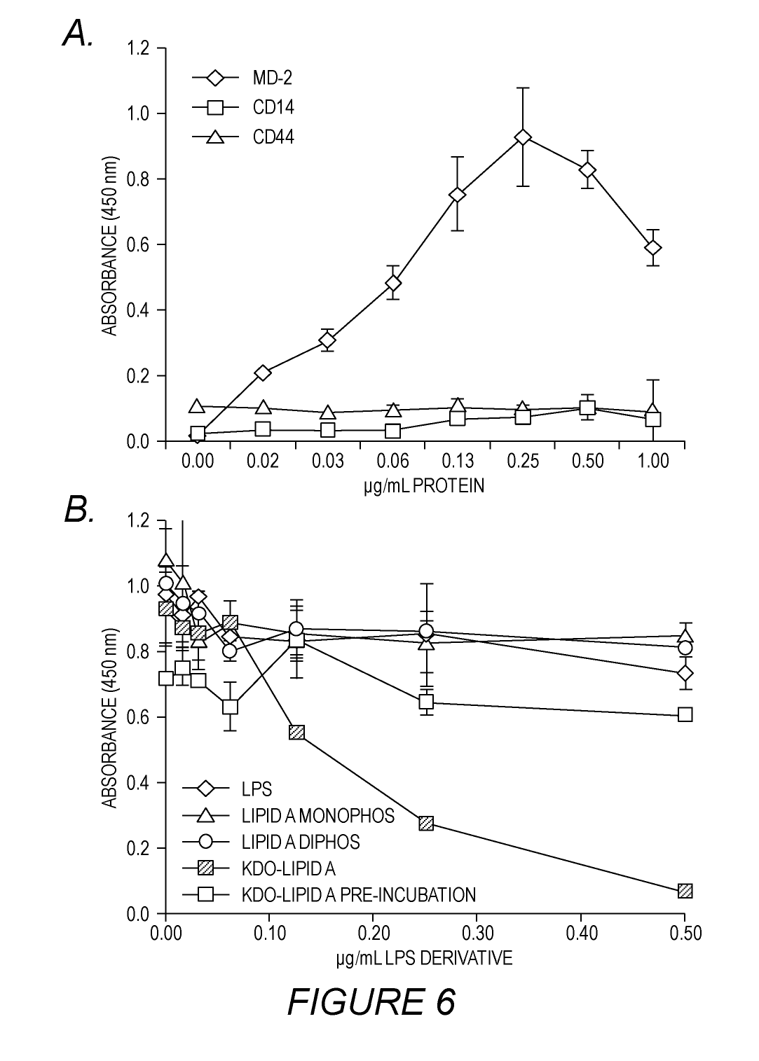

To quantitate the binding of sCD83 to MD-2, CD44, and CD14, a cell-free ELISA-based assay was developed. Increasing concentrations of recombinant MD-2, CD44s, or CD14 protein were added to immobilized sCD83 protein and the binding affinities were determined. MD-2 protein bound to sCD83 with a Kd of 7.2 nM. However, neither CD14 nor CD44s bound to sCD83 protein with a measurable affinity (FIG. 6A). The binding assay with CD44s or CD14 protein did not yield a saturation curve, indicating that sCD83 does not bind directly to CD44s or CD14.

In order to map the sCD83 binding site on MD-2, recombinant MD-2 protein was pre-incubated with each of several LPS derivatives prior to being added to plates coated with sCD83 protein. The inventors wished to test the ability of MD-2 protein to bind immobilized sCD83 protein in the presence of exogenous LPS or LPS derivatives KDO-lipid A, monophosphoryl-Lipid A, and diphosphoryl-Lipid A, as LPS-derived antagonists have been shown to prevent signaling through the TLR4/MD-2 co-receptors (Kim et al. (2007) Cell 130: 906-17). If MD-2 bound to LPS or the LPS derivatives blocked sCD83 binding, the interaction of sCD83 with MD-2 may mimic antagonist regulation of TLR signaling.

Data from these experiments is shown in FIG. 6B. Only the KDO-Lipid A variant (solid squares) prevented MD-2 binding to sCD83 when pre-incubated with MD-2 (FIG. 6B). Neither Lipid A derivatives containing one or two phosphate groups nor LPS pre-incubated with MD-2 protein blocked MD-2 binding to sCD83. However, if the KDO-lipid A variant was added first to sCD83 bound plates and then washed prior to the addition of MD-2 protein (open squares), it no longer blocked MD-2 binding to sCD83. This data indicates that sCD83 does not directly bind LPS and that LPS pre-formed with MD-2 prevents sCD83 interaction with the TLR4/MD-2 complex.

These data imply that sCD83 binds to MD-2 at a site close to the interaction of the KDO portion of the lipid molecule, but distal to the lipid A tail. Moreover, the ability of anti-CD44s and CD14 antibodies to block sCD83 cell surface binding is related to the close proximity of these receptors to TLR4. MD-2 binding to sCD83 is reminiscent of its role in the transfer of LPS from the CD14 receptor to TLR4 leading to conformational changes and initiation of signaling events. Data taken together indicate that the complex formed between MD-2 and sCD83 is stable, and MD-2 is the dominant high affinity binding partner for sCD83.

sCD83 Binding Alters the TLR4/MD-2 Signaling Cascade at the Level of IRAK-1 Protein Expression

The inventors then examined whether sCD83 acts through TLR4 to alter the signaling cascade by modulating the IL-1 Receptor-Associated Kinases (IRAK). IRAK-1 is involved in the early signaling cascade downstream of MyD88, leading to induction of NFκB and cytokine gene transcription (Ringwood and Li (2008) Cytokine 42: 1-7; Hu et al. (2002) J. Immunol. 168: 3910-3914; Li et al. (2000) J. Biol. Chem. 275: 23340-45).

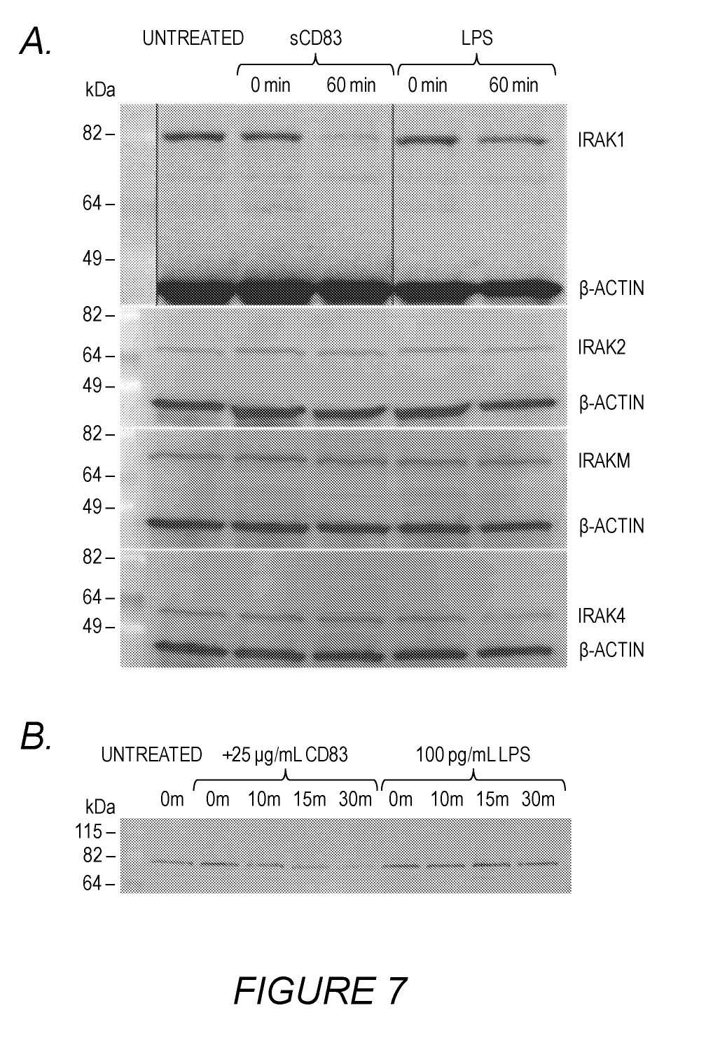

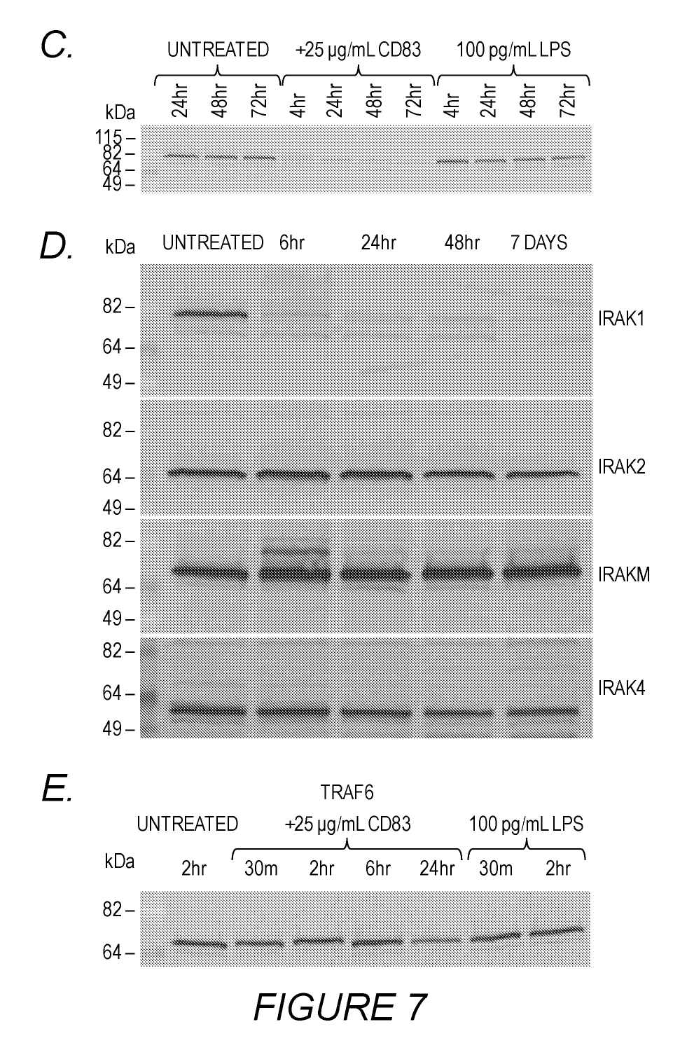

The inventors studied the expression of IRAK proteins in the presence of sCD83. Freshly isolated monocytes do not express IRAK-1 protein, even when cultured in medium alone (data not shown), so monocytes were purified by negative selection and cultured overnight in medium containing GM-CSF to induce expression of IRAK-1 protein. The GM-CSF-cultured monocytes were then incubated with sCD83 or LPS and assayed for the presence of IRAK-1, IRAK-2, IRAK-M and IRAK-4 proteins (FIG. 7). IRAK-1 protein was detected in untreated cultures and LPS-treated cultures, in contrast to cultures treated with sCD83 (FIG. 7A). Furthermore, no change in IRAK-2, IRAK-4, and IRAK-M protein levels was detected when cells were treated with sCD83 for 60 minutes.

To further study the loss of IRAK-1 protein expression within 60 minutes of treating cells with sCD83, the inventors examined IRAK-1 expression at both earlier and later time points. Data presented in FIG. 7B shows that IRAK-1 protein expression was lost rapidly by 10-15 minutes after the addition of sCD83, in contrast to the pattern shown following addition of LPS (FIG. 7B). This lack of IRAK-1 protein was sustained out past 2-3 days (FIG. 7C), and no evidence of protein restoration could be detected even at 7 days following the addition of sCD83 (FIG. 7D). As shown with the early time course, no loss of IRAK-2, IRAK-M, or IRAK-4 was seen at 2-3 and 7 days following sCD83 treatment (FIG. 7D). Also there was no impact on downstream TRAF6 protein levels during the time course of IRAK-1 loss at 30 minutes nor at 6 and 24 hours following sCD83 treatment (FIG. 7E).

sCD83 Suppresses T Cell Proliferation and Induces IL-2 Unresponsiveness

The inventors next tested the functional immune-modulatory impact of sCD83 on T cell proliferation. PBMCs containing monocytes were stimulated with anti-CD3/anti-CD28, and sCD83 was added to the cultures. The addition of soluble T-cell-specific stimulatory antibodies allowed APCs present in T cell cultures to crosslink T cell receptors and co-stimulatory molecules on the T cell, leading to T cell activation and proliferation.

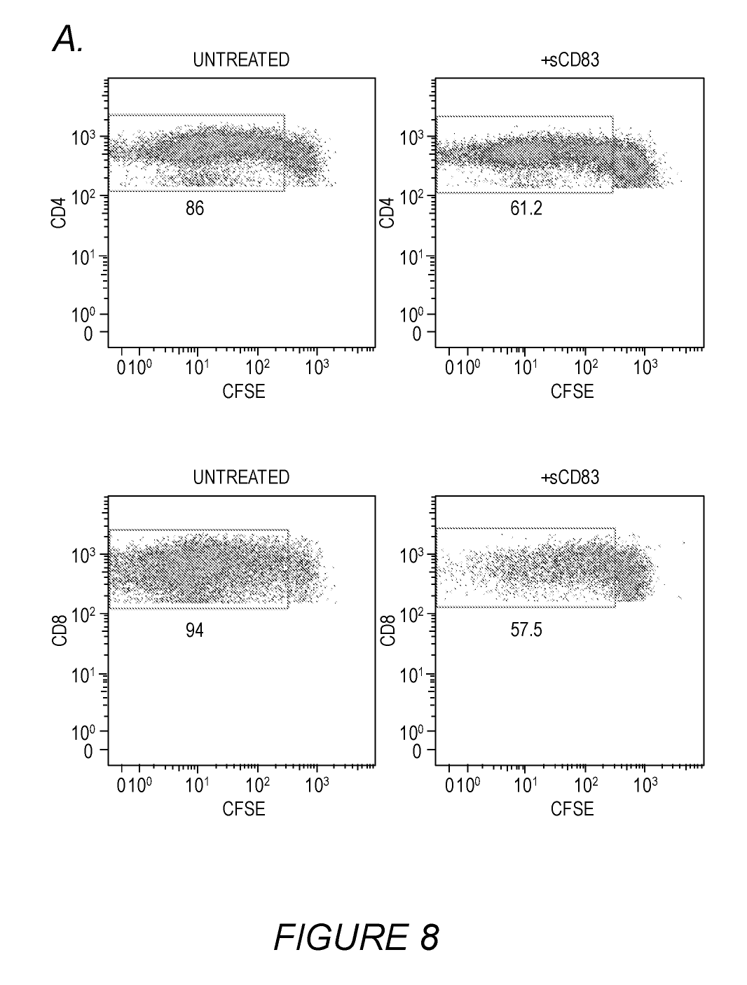

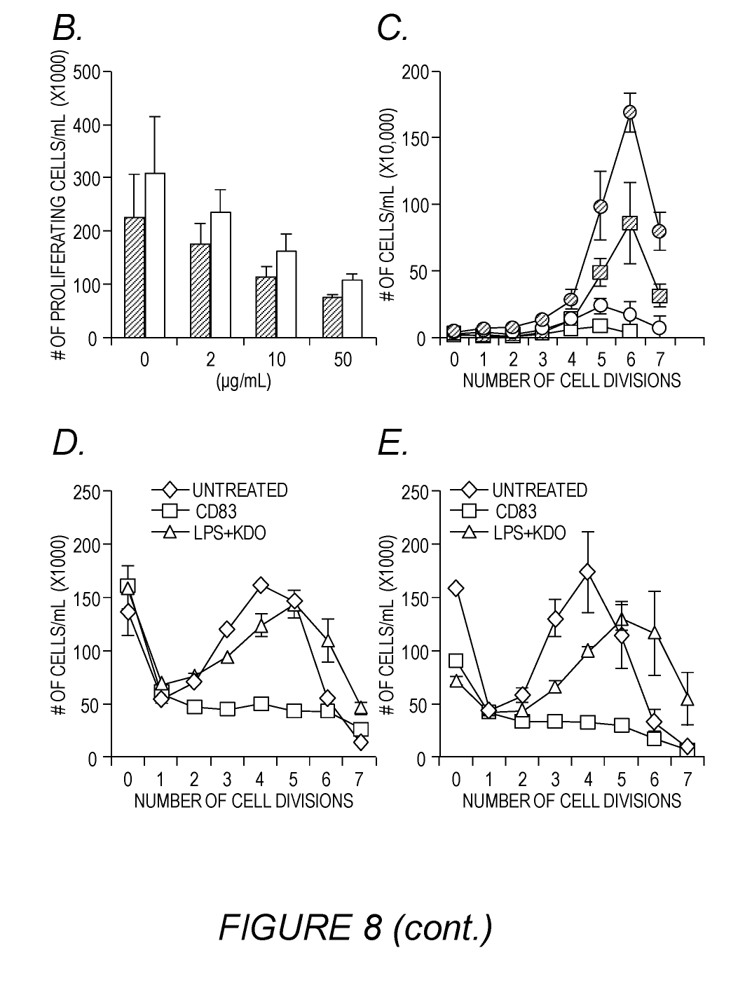

CFSE-labeled PBMCs from normal donors were stimulated in the presence of sCD83 for six days and the percentage of proliferating CD4⁺ T cells and CD8⁺ T cells were measured by CFSE dilution. sCD83 decreased CD4⁺ T cell proliferation from 86% to 61.2% and CD8⁺ T cell proliferation from 94% to 57.5% (FIG. 8A). The absolute numbers of proliferating CD4⁺ and CD8⁺ T cells were then determined for the cultures treated with sCD83. This data showed that sCD83 suppression of T cell proliferation was dose dependent, with sCD83-treated cultures having fewer total numbers of proliferating CD4⁺ and CD8⁺ T cells than the untreated cultures (FIG. 8B).

The cell populations were then assessed for their ability to expand following secondary stimulation. IL-2 was added to previously stimulated cultures of CD4⁺ and CD8⁺ T cells on day eight. Two days later, proliferation was measured by CFSE dilution over multiple cell divisions, where 0 cell divisions represents non-proliferating cells and the number of proliferating CD4⁺ and CD8⁺ T cells/mL were determined for each subsequent cell division. This data showed that both CD4⁺ and CD8⁺ T cells that were previously stimulated in the presence of sCD83 were unable to respond to IL-2 addition in comparison to T cells previously stimulated in the absence of sCD83 (FIG. 8C). This result indicates that sCD83 inhibits T cell proliferation when antigen presenting cells are present and renders T cells unresponsive to further stimulation with IL-2.

The experiments above showed that sCD83 binds to the MD-2 co-receptor of the TLR4 complex expressed on monocytes and that sCD83 addition to T cell cultures containing monocytes suppressed T cell proliferation. The inventors then examined whether LPS or KDO-Lipid A would have a similar effect as sCD83 because these molecules signal through TLR4 and may block T cell activation. These experiments were also of interest because the recombinant sCD83 protein used in the experiments above was derived from bacterial sources and could contain low levels of bacterial components such as LPS which could modulate T cell activation. To examine this issue, T cell proliferation was determined in the presence of either 50 μg/mL sCD83 or a combination of 100 ng/ml LPS and KDO-lipid A. While sCD83 suppressed both CD4⁺ and CD8⁺ T cell proliferation, the combination of LPS and KDO-lipid A did not block either CD4⁺ T cell proliferation (FIG. 8D) nor CD8⁺ T cell proliferation (FIG. 8E). These data show that sCD83 inhibition of T cell proliferation is functioning through a different mechanism than LPS signaling and that sCD83 inhibition is independent of LPS contaminants.

sCD83 Mechanism of Action is Mediated Through COX-2, IL-10 and IDO

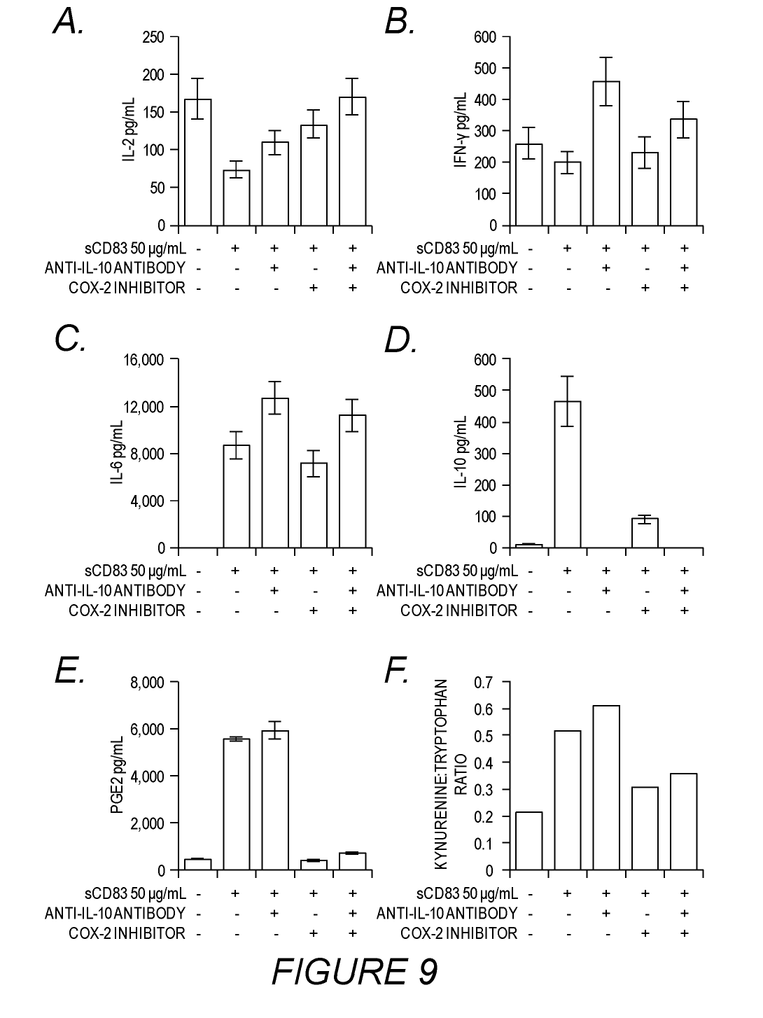

In stimulated PBMC cultures, sCD83 treatment resulted in suppression of L-2 (FIG. 9A) and to a lesser extent IFN-γ (FIG. 9B). Concurrently there was an induction of IL-6 (FIG. 9C), IL-10 (FIG. 9D) and PGE₂ (FIG. 9E). Reversal of sCD83-dependent inhibition of IL-2 was dependent on neutralization of IL-10 and blocking PGE₂ secretion with a COX-2 inhibitor (FIG. 9A). The COX-2 inhibitor “NS-398” blocked PGE₂ production (FIG. 9E) as previously reported (Chen et al. (2011) Proc. Natl. Acad. Sci. U. S. A 108: 18778-83), and in our experiments the COX-2 inhibitor suppressed sCD83 inducted IL-10 secretion (FIG. 9D) with a marginal effect on IL-6 (FIG. 9C).

### Example 2—Mapping of sCD83 Binding Sites

Experiments described above confirm that sCD83 binds to human peripheral blood monocytes (PBMCs) through interaction with the TLR4/MD-2 receptor complex. TLR4/MD-2 mediates pro-inflammatory signal delivery following recognition of bacterial lipopolysaccharides (LPS). However, sCD83 binding alters TLR4 signaling and attenuates the pro-inflammatory cascade leading to suppression of T cell activation. sCD83 specifically binds directly to MD-2 leading to this inhibition.

Tables 1-5 summarize the ability of antibodies specific for molecules or receptors associated with the TLR4/MD-2 signaling complex that block the binding of soluble CD83 to the TLR4/MD-2 complex of receptors. These molecules or receptors include CD14, CD44 and its variant forms, CD11b, and CD11a. Thus, antibodies to different molecules/receptors reported to associate with the TLR complex can block sCD83 binding, including variants of LPS, demonstrating that these molecules are in close proximity to the sCD83 binding site on the TLR4 complex. Bispecific antibodies that bind to regions of these molecules that are associated with the sCD83 binding site or are the sCD83 binding site could mimic the effects of conformational or structural changes induced as a consequence of sCD83 binding. In this manner, bispecific antibodies could induce anti-inflammatory signals or tolerogenic signals through the TLR4 receptor similar to the role played by sCD83.

TABLE 1
Summary of antibody blocking sCD83-m3 binding to post-thaw PBMCs
Blocking sCD83-m3 binding on monocytes*

| | % inhibition (relative |
|---|---|
| Antibody | to control) |
| anti-CD18 | 98% |
| anti-CD18 (MEM-148) | No blocking |
| anti-CD14 (My4) | 84% |
| anti-CD11a | 53% |
| anti-CD11b | 92% |
| anti-CD11c | No blocking |
| anti-TLR2 | No blocking |
| anti-TLR4 | No blocking |
| anti-MD-1 | No blocking |
| soluble LPS | 5% |

*91% of cells bind sCD83 without any blocking (control)
Anti-CD18 clone MEM-148 detects a truncated form of CD18 which lacks the ability to associate with CD11a or CD11b.
Anti-CD14 clone My4 blocks the LPS binding site on CD14.

TABLE 2
Summary of antibody blocking of sCD83-m3
binding to cultured PBMCs

| | Percent inhibition of: | | |
|---|---|---|---|
| | Post thaw PBMC | Cultured PBMC | LPS/GMCSF |
| | sCD83 binding | binding | stimulated PBMC |
| Antibody | (=73%) | (=57%) | 72 hrs |
| anti-CD14 | 97% | 54% | 100% |
| anti-CD11b | 100% | 1% | No blocking |
| anti-MD-2 | 81% | 31% | |

Activation of monocytes results in the loss of anti-CD11b blocking of sCD83. However, anti-CD14 blocking is retained. Antibodies directed to MD-2 of the TLR4 complex block sCD83 binding.

TABLE 3
LPS derivatives pre-incubated with MD-2; loss
of MD-2 binding to sCD83-m3 (cell free ELISA)
% inhibition of MD-2 binding to sCD83-m3

| | | Lipid A | Lipid A |
|---|---|---|---|
| LPS Ultra Pure | KDO | Monophos | Diphos |
| 25% | 93% | 22% | 19% |

sCD83 protein will bind MD-2 protein in a cell free ELISA. KDO (keto-deoxyoctulosonate) is the sugar core of LPS; only the KDO portion of LPS blocks sCD83 binding to MD-2.

TABLE 4
Anti-CD44 antibody blocking of sCD3-m3 binding

| | sCD83-m3 + Anti- | |
|---|---|---|
| sC83D-m3 binding | CD44v6 (clone VFF-6) | |
| no blocking antibodies | block | % inhibition |
| 57.3% | 14.3% | 75% |
| sCD-m3 binding no | sCD83-m3 + Anti- | |
| blocking | CD44s block | % inhibition |
| 65.5% | 2.06% | 97% |

Antibodies to CD44 containing at least the region encoded by variant 6 block sCD83 binding to monocytes

TABLE 5

| antibody clones | |
|---|---|
| BD ® | |
| Biosciences | |
| CD14 | M5E2 |
| CD44 | 515 |
| CD11b | ICRF44 |
| CD80 | L307.4 |
| HLA-A, B, C | DX17 |
| eBioscience ® | |
| Corp. | |
| CD14 | 61D3 |
| MD2 | 9B4 |
| TLR4 | HTA125 |
| CD44 | IM7 |
| CD44v6 | VFF-18 |
| CD44v6 | VFF-7 |
| CD44v6 | VFF-6 |
| Invitrogen ® | |
| Corp. | |
| CD44 | 156-3c11 |
| CD44 | 5F12 |
| R&D | |
| Systems ®, Inc. | |
| CD83 | Polyclonal |
| MD2 | 288307 |
| Beckman | |
| Coulter ®, Inc. | |
| CD14 | My4 |

### Example 3—IRAK Assay of CD83 Activity

Applicants discovered that sCD83 induces rapid down-regulation of IRAK-1 protein in macrophage cultures, whereas LPS does not have this effect. This assay (“IRAK assay”) can thus be used to distinguish between preparations/formulations that have CD83 activity and those that do not.

### Cell Culture

One vial (2×10⁸ total cells) of 2124557 PBMCs were thawed and cultured in T-150 flask with 1000 U/mL GM-CSF for 5 days. The macrophages were harvested, counted and resuspended at 1×10⁶ cells/mL in R-10 media with GM-CSF and then cultured overnight. The cells were incubated with various preparations of sCD83 polypeptides, +/−25 μg/mL in 1 mL cultures for various times and then lysed in ice cold RIPA buffer with protease/phosphatase inhibitor cocktail. Bradford (BCA) protein assay was performed to determine total protein concentration.

### Gel and Western Blot

4-5 μg of each lysate sample was run on a lane of a Criterion 10-20% Tris-HCL gel at 200V for 50 minutes. The gel was transferred to PVDF membrane at 100V for 75 minutes. The PVDF membrane was washed in Tris-Buffered Saline with Tween 20 (TBS-T) and then blocked in 3% nonfat milk in TBS-T for 1 hour and washed 3× in TBS-T. The washed membrane was incubated with primary antibody (1:1000): anti IRAK1 antibody in 3% BSA in TBS-T at 4° C. overnight. The membrane was washed, then incubated with anti-rabbit HRP in blocking buffer for 1 hr. The blot was then developed using ECL Plus kit.

Results of one experiment showing the distinguishing abilities of the IRAK assay demonstrated a positive result for active CD83 and a negative result for LPS.

### Example 4—Macrophage Assay for CD83 Activity

Other assays of CD83 activity were previously known in the art but were inadequate for quickly and efficiently assessing CD83 activity; for example, the “TNF a assay” (for example as described in Example 4 of WO 2009/142759) showed sensitivity to contaminants of sCD83 preparations, including the common contaminant LPS. Animal transplantation assays could also be used but were relatively very expensive and time-consuming. Thus, there was a need for quick, reliable, in vitro assays for CD83 activity to make experimentation on variants and formulations of CD83 economically and practically feasible. Accordingly, Applicants developed the following “macrophage assay” for CD83 activity, which measures the polarization of pro-inflammatory macrophages (M1) to anti-inflammatory macrophages (M2).

### Materials and Methods: Generation of Macrophages

Leukapheresis from healthy volunteers was provided by Keybiologics (Memphis, Tenn.). Mononuclear cells were isolated using Histopaque®-1077 reagent (Sigma-Aldrich® Corporation), re-suspended in Fetal Bovine Serum (Atlanta Biologicals) containing 10% DMSO (Sigma-Aldrich® Corporation) and stored in liquid nitrogen. Thawed cells (2×10⁸ cells) were washed once in PBS (Cambrex™ Corp.) and cultured in 30 ml X-VIVO 15 (Cambrex™ Corp.) medium in Corning® T150 flasks for 5 days in a 5% CO₂ incubator at 37° C., supplemented with 1000 U/ml of GM-CSF (Leukine liquid, Berlex® Laboratories, Inc.). After 5 days, pro-inflammatory (M1) macrophages were harvested. Non-adherent cells were decanted into 50 ml tubes. Flasks were rinsed twice with PBS, re-suspended with cold X-VIVO 15 and incubated for 30 minutes at 4 degrees Celsius. Adherent cells were removed using a cell scraper (BD® Bioscience) and added back to non-adherent cells or processed alone. Cells were centrifuged and re-suspended at 1×10⁶ cells/ml in X-VIVO 15 supplemented with 1000 U/ml of GM-CSF. Cells were transferred to low adherence Corning® 24 well plates at 1×10⁶ cells/ml, treated with or without 50 μg of soluble CD83, and cultured in a 5% CO₂ incubator at 37° C. for 3 or 4 days.

### Macrophage Surface Antigen Expression

Macrophage differentiation was assessed using monoclonal antibodies conjugated with FITC, PE, or APC fluorochrome directed against various cell surface antigens. Mouse monoclonal antibodies to the following antigens were used: CD11b, CD14, CD16, CD25, CD38, CD80, CD83, CD86, CD209, HLA-DR (BD® Biosciences), and CD163 (eBioscience® Corp.). Antibodies were incubated with 100 μl of cells re-suspended in BD® Stain Buffer (FBS) for 15 minutes at room temperature. Cells were washed once with 2 ml BD® Stain Buffer (FBS), centrifuged and re-suspended in 0.5 ml BD® Stain Buffer (FBS) containing 7-AAD (BD® Bioscience) for dead cell exclusion and acquired on a FACSCalibur™ Flow Cytometer (BD® Bioscience). Flow Cytometry analysis was performed using FlowJo® software (Tree Star, Inc) with a macrophage gate set using forward and side scatter parameters.

In one set of assays, a particular preparation of soluble CD83 was evaluated for effects on GM-CSF treated monocyte-macrophage surface antigen expression. Monocytes treated with GM-CSF were cultured in serum-free medium for 5 days. Bulk cultures with 67% lymphocytes or enriched macrophages with 4% lymphocytes were treated with soluble CD83 or none for 4 days. Bulk cultures treated with soluble CD83 showed up-regulation of CD163, CD16, and CD38. Enriched monocytes showed partial up-regulation of antigen while untreated bulk cultures showed no up-regulation of antigen.

These experiments demonstrated specific interaction of soluble CD83 with mature macrophages derived from GM-CSF treated monocytes in serum-free culture conditions. Pro-inflammatory generated macrophages (M1) were polarized to anti-inflammatory macrophage (M2) over a 3 or 4 day period after exposure to active soluble CD83 defined by phenotypic changes. This effect of soluble CD83 in the polarization of M1 to M2 macrophages was not related to contaminants such as endotoxin, enterotoxin or thrombin. Thus, an increase in expression of CD163, CD16, and/or CD38 by at least 10%, 20%, 30%, 40%, 50%, 60%, 70%, or 100% or more in a population of cells in this assay indicates a positive result for sCD83 activity.

We claim:

1. A bispecific antibody comprising at least one anti-MD-2 antibody or antigen-binding fragment thereof and at least one anti-CD44 antibody or antigen-binding fragment thereof.

2. The antibody of claim 1, wherein said at least one anti-MD-2 antibody or antigen-binding fragment thereof competes with sCD83-m3 for binding to MD-2.

3. The antibody of claim 1, wherein said at least one anti-MD-2 antibody or antigen-binding fragment thereof competes with KDO for binding to MD-2.

4. The antibody of claim 1, wherein said at least one anti-CD44 antibody or antigen-binding fragment thereof competes for CD44 binding with the reference antibody VFF-6, VFF-7, or VFF-18.

5. The antibody of claim 4, wherein said at least one anti-CD44 antibody or antigen-binding fragment thereof does not compete for CD44 binding with the reference antibody 1M7.

6. The composition of claim 1, wherein said antibody competes for binding to MD-2 with the reference antibody 288307.

7. The composition of claim 6, wherein said antibody does not compete for binding to MD-2 with the reference antibody 9B4 and/or MTS510.

8. A method for decreasing the amount of IRAK-1 produced by a cell comprising exposing said cell to the antibody of claim 1.

9. A bispecific antibody comprising at least one anti-MD-2 antibody or antigen-binding fragment thereof and at least one anti-CD14 antibody or antigen-binding fragment thereof.

10. A method for decreasing the amount of IRAK-1 produced by a population of cells comprising exposing said population of cells to the antibody of claim 1.

11. A method for increasing the amount of IL-10 secreted by a cell expressing CD14 on its cell surface comprising the step of exposing said cell to the antibody of claim 11.

12. A method of treating or preventing an unwanted immune response in a mammalian subject, comprising administering the antibody of claim 1 to said subject.

13. The method of claim 12, wherein the unwanted immune response is selected from the group consisting of autoimmune diseases, transplant rejection, and allergy.

14. The method of claim 13, wherein the autoimmune disease is selected from the group consisting of systemic lupus erythematosus, type I diabetes, Pemphigus, Grave's disease, Hashimoto's thyroiditis, myasthenia gravis, automyocarditis, multiple sclerosis, rheumatoid arthritis, psoriasis, autoimmune uveoretinitis, vasculitis, a chronic inflammatory bowel disease such as Crohn's disease or ulcerative colitis, Morbus Bechterew, ankylosing spondylitis and chronic obstructive pulmonary disease (COPD).
